(19) United States
(12) Patent Application Publication
(10) Pub. No.: US 2023/0277741 A1
(43) Pub. Date: Sep. 7, 2023

(54) FLUID CONTROL AND BYPASS FEATURES FOR AN APHERESIS SYSTEM

(71) Applicant: Terumo BCT, Inc., Lakewood, CO (US)

(72) Inventors: Bruce ELLINGBOE, Littleton, CO (US); Anne Baker ORAM, Lakewood, CO (US); Hong Duc NGUYEN, Lakewood, CO (US); Evan Christopher PROVAN, Littleton, CO (US); Mark Evan HILLAM, Wheat Ridge, CO (US); Charles L. HAKE, Arvada, CO (US)

(21) Appl. No.: 18/116,527

(22) Filed: Mar. 2, 2023

Related U.S. Application Data
(60) Provisional application No. 63/318,735, filed on Mar. 10, 2022.
(60) Provisional application No. 63/317,489, filed on Mar. 7, 2022.

(51) Int. Cl. A61M 1/34; A61M 39/10

(52) U.S. Cl. CPC A61M 1/3496; A61M 39/10

(57) ABSTRACT

A pump for fluids includes a rotor sub-assembly, a tubing pressure block, an inlet guide, an outlet guide, and a tubing guard. The rotor sub-assembly includes at least one roller. The tubing pressure block includes a raceway and at least one projection. The tubing pressure block is movable between a first position and a second position. The inlet guide includes an inlet tubing channel and is disposed proximate to a first side of the tubing pressure block. The outlet guide includes an outlet tubing channel and is disposed proximate to a second side of the tubing pressure block. The tubing guard is configured to engage with the inlet guide and the outlet guide when the tubing guard is in a closed position and is configured to expose the rotor sub-assembly, the tubing pressure block, the inlet guide, and the outlet guide when in an open position.

### CROSS-REFERENCE TO RELATED APPLICATION

This application claims the benefit of U.S. Provisional Application No. 63/317,489 filed on Mar. 7, 2022 and U.S. Provisional Application No. 63/318,735 filed on Mar. 10, 2022. The entire disclosures of each of which are incorporated herein by reference.

### FIELD

The present disclosure is generally directed to fluid control and bypass features, in particular, within apheresis systems.

### BACKGROUND

The present disclosure is generally directed to separating components from multi-component fluids, in particular, toward apheresis methods and systems.

There are two common methods for blood donation/collection. The first is whole blood donation from a donor, followed by a centrifugal process that separates blood components from the whole blood based on the density of the blood component. The desired component can be manually, semi-automatically, or automatically moved to a collection container during, or possibly, after the whole blood is under the effect of the forces produced by the centrifuge. The other method may be an apheresis collection that requires a specialized machine.

The apheresis method extracts whole blood from a donor while the donor is connected to the specialized machine. The whole blood can again be centrifuged to collect only the blood component (e.g., plasma) that is desired and can return all other blood components not desired back to the donor during the same donation. The donor is connected to the apheresis machine during the separation and collection of the blood component. Unfortunately, the apheresis process can be lengthy and uncomfortable. Often, the donor must remain connected to the machine for an hour to obtain a blood component donation. Thus, making the donation procedure more efficient is an ongoing desire for apheresis collection sites.

### SUMMARY

There is a need for a plasma or other blood component system that can reduce the donation time and increase the comfort of the donor. Embodiments presented herein can increase the efficiency of the donation process by using the separated blood component to push or drive the non-desired blood components back to the donor without stopping and restarting the centrifuge. Thus, the embodiments herein make the donation process more efficient and faster for the donor.

Embodiments may also provide methods and apparatuses for positioning portions, e.g., loops, of disposables in medical devices. Embodiments may involve use of surfaces for automatically guiding loops. In at least one example embodiment, the medical devices may be blood separation machines, such as apheresis machines.

The previously mentioned and other needs are addressed by the various aspects, embodiments, and/or configurations of the present disclosure. Also, while the disclosure is presented in terms of exemplary embodiments, it should be appreciated that individual aspects of the disclosure can be separately claimed.

In at least one example embodiment, a pump for fluids is described. The pump may include a rotor sub-assembly, a tubing pressure block, an inlet guide, an outlet guide, and a tubing guard. The rotor sub-assembly may include at least one roller. The tubing pressure block may include a raceway and at least one projection. The tubing pressure block may be movable between a first position and a second position. The inlet guide may include an inlet tubing channel and may be disposed proximate to a first side of the tubing pressure block. The outlet guide may include an outlet tubing channel and may be disposed proximate to a second side of the tubing pressure block. The second side of the tubing pressure block may be disposed opposite the first side of the tubing pressure block such that there is a substantially straight path between the inlet guide and the outlet guide. The tubing guard may be configured to engage with the inlet guide and the outlet guide when the tubing guard is in a closed position and may be configured to expose the rotor sub-assembly, the tubing pressure block, the inlet guide, and the outlet guide when in an open position.

In at least one example embodiment, the raceway may be curved and may be configured to meet an arc of the rotor sub-assembly when the tubing pressure block is in the first position.

In at least one example embodiment, the pump may be configured to engage a section of tubing. The section of tubing may be disposed through the inlet guide, the raceway, and the outlet guide. In at least one example embodiment, the section of tubing may be configured to be semi-occluded when the tubing pressure block is in the second position. In at least one example embodiment, the at least one projection and the at least one roller are configured to engage with the section of tubing to cause the tubing to be semi-occluded when the tubing pressure block is in the second position. In at least one example embodiment, the tubing guard may include at least one channel projection configured to engage with at least one of the inlet channel or the outlet channel when the tubing guard is in the closed position. In at least one example embodiment, the at least one channel projection may be configured to engage with and semi-occlude the section of tubing when the tubing guard is in the closed position. In at least one example embodiment, the at least one channel projection includes a first channel projection configured to engage with the inlet channel and a second channel projection configured to engage with the outlet channel. In at least one example embodiment, the section of tubing may be configured to stretch when the pump is in operation. The raceway may include at least one sidewall feature configured to collect a stretched portion of the section of tubing.

In at least one example embodiment, the pump may further include at least one sensor disposed proximate to at least one of the inlet guide, the outlet guide, or the tubing pressure block. In at least one example embodiment, the at least one sensor may include at least one of a pressure sensor, a line sensor, a cover position sensor, a movable block position sensor, an inductive sensor, an optical sensor, a light sensor, an ultrasonic sensor, or an air or fluid sensor.

In at least one example embodiment, the tubing pressure block may include a cavity with at least one bias member configured to maintain the tubing pressure block in at least one of the first position or the second position. In at least one example embodiment, the at least one bias member may be at least one spring. In at least one example embodiment, the tubing pressure block may further include at least one driven motive member disposed within the cavity. The at least one driven motive member may be configured to overcome the at least one bias member. In at least one example embodiment, the at least one driven motive member may be a pneumatic diaphragm. The pneumatic diaphragm may be configured to inflate and move the tubing pressure block away from the first position to the second position. In at least one example embodiment, the pump may be a normally closed pump, the first position may be a closed position, the second position may be an open position, the at least one bias member may be configured to maintain the tubing pressure block in the first position, and the at least one driven motive member may be configured to overcome the at least one bias member to move the tubing pressure block to the second position. In at least one example embodiment, the pump may be a normally open pump, the first position may be a closed position, the second position may be an open position, the at least one bias member may be configured to maintain the tubing pressure block in the second position, and the at least one driven motive member may be configured to overcome the at least one bias member to move the tubing pressure block to the first position. In at least one example embodiment, the pump may be an anticoagulant pump and the first position may be a closed position. In at least one example embodiment, the tubing pressure block may be configured to move from the first position to the second position when an external force is applied to the tubing pressure block.

In at least one example embodiment, the pump may further include a fluid ingress prevention feature configured to collect and prevent fluid from contacting at least one internal pump component.

Also described herein is a method for fluid control in a pump. The method may include inserting tubing into a pump via an inlet guide and an outlet guide. The pump may be in an open state when the tubing is inserted with a tubing pressure block in a first position. The method may further include closing the pump by moving the tubing pressure block to a second position. The tubing may be fully occluded between at least one roller of a rotor sub-assembly and a raceway of the tubing pressure block when the tubing pressure block is in the second position. The method may additionally include actuating the pump by rotating the at least one roller such that fluid within the tubing is moved in a direction corresponding to rotation of the rollers.

In at least one example embodiment, the pump may include a tubing guard configured to engage with the inlet guide and the outlet guide. In at least one example embodiment, the tubing may be disposed between the tubing guard and the inlet guide and between the tubing guard and the outlet guide when the pump is closed. In at least one example embodiment, the tubing may be clamped into a diamond shape when disposed between the tubing guard and the inlet guide and between the tubing guard and the outlet guide.

In at least one example embodiment, the tubing may be configured to expand when the pump is in operation and the inlet guide and the outlet guide include at least one cut-out configured to house the expanded tubing when the pump is in operation.

Also described herein is a method of fluid control through an apheresis system. The method may include actuating a first pump to draw whole blood from a donor, receiving whole blood from the donor via an inlet tubing fluidly connected to and within the apheresis system, moving the whole blood through the first pump of the apheresis system, stopping the first pump, and actuating a second pump to move at least one component of the whole blood to a collection component of the apheresis system. In at least one example embodiment, actuating the second pump may include moving the second pump from an open state to a closed state. In at least one example embodiment, when the second pump is actuated, the first pump is moved from a closed state to an open state.

In at least one example embodiment, the apheresis system may include a third pump that may be configured to be in a closed state while the apheresis system is operational.

Embodiments include a method for collecting a blood component through apheresis, the method comprising: drawing whole blood into a centrifuge from a donor; spinning the centrifuge to cause centrifugal force to act on the whole blood to separate the whole blood into a least a first blood component and red blood cells; separating a first blood component from the whole blood; extracting the first blood component into a container; detecting when a second blood component is being extracted; and after the second blood component is detected and while the centrifuge continues to spin, forcing the separated first blood component back towards the centrifuge to move at least the red bloods cells from the centrifuge and back into the donor.

Aspects of the above method include wherein the first blood component is one or more of plasma, platelets, red blood cells and/or high hematocrit blood. Aspects of the above method include wherein the second blood component is one or more of plasma, platelets, red blood cells and/or high hematocrit blood. Aspects of the above method include wherein the first blood component is two or more of plasma, platelets, red blood cells and/or high hematocrit blood. Aspects of the above method include wherein the centrifuge spins at a first speed when separating the first blood component from the whole blood. Aspects of the above method include wherein the centrifuge continues to spin at the first speed when forcing the separated first blood component back towards the centrifuge. Aspects of the above method include wherein the centrifuge spins at a second speed when drawing whole blood into the centrifuge from the donor. Aspects of the above method include wherein the second speed is slower than the first speed. Aspects of the above method include wherein the first blood component is separated from the whole blood in a blood component collection set that is inserted into the centrifuge. Aspects of the above method include wherein the centrifuge includes a filler that spins a blood component collection bladder associated with the blood component collection set. Aspects of the above method include wherein the blood component collection bladder is inserted into a collection insert channel formed in the filler to hold the blood component collection bladder.

Embodiments include an apheresis system comprising: a needle inserted into a blood vessel of a donor to draw whole blood from the donor; a first tube having a lumen, fluidly associated with the needle, that moves the whole blood through the lumen; a draw pump engaged with the first tube that draws the whole blood into a centrifuge from the donor; the centrifuge that spins to cause centrifugal force to act on the whole blood to separate the whole blood into a least a first blood component and red blood cells; a blood component collection bladder, inserted into the centrifuge and fluidly associated with the first tube, that separates the first blood component from the whole blood; a second tube, fluidly associated the blood collection bladder, that moves the first blood component from the blood component collection bladder; a collection bottle, fluidly associated with the second tube, that extracts the first blood component from the apheresis system; a sensor positioned in physical proximity to the second tube to detect when a second blood component is being extracted from the whole blood; and after the second blood component is detected by the sensor and while the centrifuge continues to spin, a return pump, engaged with the second tube, that forces the separated first blood component back towards the blood component collection bladder through the second tube to move at least the red bloods cells from the blood component collection bladder and back into the donor.

Aspects of the above apheresis system include wherein the first blood component is plasma and the second blood component is platelets, red blood cells, and/or high hematocrit blood. Aspects of the above apheresis system further comprises an anticoagulant pump to draw anticoagulant from an anticoagulant bag and mix the anticoagulant with whole blood at a manifold or junction fluidly associated with the first tube. Aspects of the above apheresis system include wherein the centrifuge includes a filler that spins the blood component collection bladder. Aspects of the above apheresis system include wherein the blood component collection bladder is inserted into a collection insert channel formed in the filler to hold the blood component collection bladder.

Embodiments include a blood component collection set associated with an apheresis system comprising: a needle inserted into a blood vessel of a donor to draw whole blood from the donor; a first tube having a lumen, fluidly associated with the needle, that moves the whole blood through the lumen, wherein a draw pump engaged with the first tube draws the whole blood from a donor; a blood component collection bladder, inserted into a centrifuge and fluidly associated with the first tube, that separates the first blood component from the whole blood; a second tube, fluidly associated with the blood collection bladder, that moves the first blood component from the blood component collection bladder; and a collection bottle fluidly associated with the second tube that extracts the first blood component from the apheresis system, wherein a sensor is positioned in physical proximity to the second tube to detect when a second blood component is being extracted from the whole blood; and wherein, after the second blood component is detected by the sensor and while the centrifuge continues to spin, a return pump engaged with the second tube forces the separated first blood component back towards the blood component collection bladder through the second tube to move at least the red bloods cells from the blood component collection bladder and back into the donor.

Aspects of the above blood component collection set include wherein the first blood component is plasma and the second blood component is platelets. Aspects of the above blood component collection set include wherein the draw pump is disengaged when the return pump forces the separated first blood component back towards the blood component collection bladder through the second tube to move at least the red bloods cells from the blood component collection bladder and back into the donor. Aspects of the above blood component collection set include wherein the blood component collection bladder is inserted and held in a filler, in the centrifuge, that spins the blood component collection bladder. Aspects of the above blood component collection set include wherein the blood component collection bladder is inserted into a collection insert channel formed in the filler to hold the blood component collection bladder.

Aspects of the above method include wherein, when drawing fluid from the donor in a subsequent draw, a portion of the fluid previously sent to the donor through the direct flow lumen, when returning red blood cells to the donor, is maintained in the drip chamber when the whole blood again passes through the fluid flow bypass path.

Embodiments include a pump for one or more of biological, medical or intravenous fluids comprising: a base member; a rotor sub-assembly having at least one roller, the rotor sub-assembly being disposed connected to and supported by the base member, and the rotor sub-assembly defining a partial arc disposed to operatively engage a portion of tubing operatively engaged thereby; a movable pressure block having a race surface disposed thereon; and at least one projection to engage and partially deform but not fully occlude a portion of tubing adjacent the rotor, the movable pressure block being movable to alternately engage and disengage the at least one projection and the race surface into or away from operative disposition relative to the rotor sub-assembly and the at least one roller at the partial arc of the rotor sub-assembly; an inlet guide member and an outlet guide member, the inlet guide member having an inlet tubing channel formed therein, the outlet guide member having an outlet tubing channel formed therein, the inlet guide member and the outlet guide member being disposed on opposite sides of the moveable pressure block and the inlet tubing channel and the outlet tubing channel forming substantially a linear form for receiving a tubing section in a substantially straight line; a moveable cover sub-assembly rotatably connected to the inlet and outlet guide members and operatively disposed to cover the operative engagement of the race surface and the arc of the rotor sub-assembly and a portion of each of the inlet channel and a portion of the outlet channel adjacent the race surface and the arc of the rotor sub-assembly; the moveable cover sub-assembly having at least one channel projection to enter a portion of either one or the other of the inlet and outlet channels to engage and partially deform but not fully occlude a portion of tubing adjacent the rotor.

Aspects of the above include the raceway being curved to meet the arc of the rotor sub-assembly. Aspects of the above include the inlet channel, the outlet channel, the raceway and the rotor defining a substantially straight tubing loading disposition but for the curved portion of the raceway and the arc of the rotor sub-assembly where the raceway operably engages with the rotor sub-assembly. Aspects of the above include the at least one projection of the moveable pressure block providing a tortuous fluid path within a tubing portion engaged thereby. Aspects of the above include the at least one projection of the moveable cover member providing a tortuous fluid path within a tubing portion engaged thereby. Aspects of the above include the at least one projection of the moveable pressure block including first and second projections, the first projection being defined adjacent the inlet channel of the inlet guide member and the second projection being defined adjacent the outlet channel of the outlet guide member, both the first and second projections providing first and second tortuous fluid paths within a tubing portion engaged thereby. Aspects of the above include the at least one projection of the moveable cover member including at least first and second projections, the first projection being defined operatively insertable within the inlet channel of the inlet guide member and the second projection being defined operatively insertable within the outlet channel of the outlet guide member, both the first and second projections providing first and second tortuous fluid paths within a tubing portion engaged thereby. Aspects of the above include the at least one projection of the moveable cover member including at least a third projection, the third projection being defined operatively insertable within one of the inlet channel of the inlet guide member and the outlet channel of the outlet guide member, the third projection providing a third tortuous fluid path within a tubing portion engaged thereby. Aspects of the above include one of the inlet channel and the outlet channel having at least a first recess, the first recess being defined to operatively receive the at least one projection of the moveable cover member insertable within one of the inlet channel of the inlet guide member and the outlet channel of the outlet guide member, the first recess providing a tortuous fluid path within a tubing portion engaged thereby. Aspects of the above include the inlet channel or the outlet channel having one or more of at least a second recess and a third recess, the second and third recesses being defined to operatively receive the at least one projection of the moveable cover member insertable within one of the inlet channel of the inlet guide member and the outlet channel of the outlet guide member, the second and third recesses providing a tortuous fluid path within a tubing portion engaged thereby. Aspects of the above include the at least one projection of at least one of the movable pressure block and the movable cover sub-assembly minimizing one or both of: longitudinal movement of a tubing portion in the inlet channel, the outlet channel or the raceway; and, lateral movement of a tubing portion in the inlet channel, the outlet channel or the raceway.

Aspects of the above include a system further comprising at least one sensor disposed in or adjacent one of the inlet channel or the outlet channel or the movable pressure block raceway. Aspects of the above include at least one sensor disposed in or adjacent one of the inlet channel or the outlet channel. Aspects of the above include the at least one sensor comprising two or more sensors. Aspects of the above include the at least one sensor comprising one or more of pressure sensors, line sensors, cover position sensors, movable block position sensors, inductive sensors, optical sensors, light sensors, ultrasonic sensors, or air or gas sensors. Aspects of the above include the at least one projection of at least one of the movable pressure block and the movable cover sub-assembly minimizing movement of a tubing portion relative to the at least one sensor.

Embodiments include a method for minimizing movement of a tubing section in a pump for one or more of biologic, medical or intravenous fluids, the method comprising: engaging a portion of a tubing section with a projection of at least one projection of at least one of a movable pressure block and a movable cover sub-assembly minimizing movement of a tubing portion in one of an inlet channel, an outlet channel and a raceway; reducing movement relative to one or both: a pump roller, and a sensor.

Aspects of the above include the engaging being one or more of: crimping the tubing; and partially occluding the tubing; and providing a tortuous fluid path within the tubing portion engaged thereby. Aspects of the above include the reducing being one or more of: reducing movement of the tubing longitudinally; reducing movement of the tubing laterally; reducing movement for increased rotor and pumping efficiency; and, reducing movement for increased sensor operability. Aspects of the above include the reducing including one or more of: longitudinally being defined along the length of one or more of the inlet channel, the outlet channel, and the raceway; longitudinally being defined along the length of a curved surface of the raceway; laterally being defined along cross-wise relative to the length of a tubing section; laterally being defined along cross-wise relative to the length of a tubing section at one or the other of a right angle or an angle oblique or acute to a right angle.

Embodiments include a pump for one or more of biological, medical or intravenous fluids that is one of Normally Open or Normally Closed; the pump comprising: a base member; a rotor sub-assembly having at least one roller, the rotor sub-assembly being disposed connected to and supported by the base member, and the rotor sub-assembly having a partial arc disposed to operatively engage a portion of tubing operatively engaged thereby; a movable pressure block having a race surface disposed thereon to contact with the rotor sub-assembly to fully occlude a portion of tubing adjacent the rotor, the movable pressure block being movable to alternately engage and disengage the at least one projection and the race surface into or away from operative disposition relative to the rotor sub-assembly and the at least one roller at the partial arc of the rotor sub-assembly; the movable pressure block having a cavity defined therein, within which are disposed: at least one bias member disposed within the cavity to hold the movable pressure block in one of the Normally Open position or Normally Closed position; and, a driven motive member operatively disposed within the cavity to overcome the holding of the bias member and move the movable pressure block away from the biased Normally Open position or Normally Closed position.

Aspects of the above include the bias member being: at least one spring to bias to one of the Normally Open position or the Normally Closed position. Aspects of the above include the driven motive member being: a pneumatic diaphragm operatively disposed within the cavity to inflate and move the movable pressure block away from the biased Normally Open or Normally Closed position. Aspects of the above include the movable block having: a forward internal wall defined within the cavity, the forward wall being disposed near the raceway surface; and a rearward internal wall defined within the cavity defined further from the raceway surface than the forward internal wall; a support wall operatively associated with and disposed within but not structurally connected to the movable block, the support wall not being movable relative to the movability of the movable pressure block; the bias member being disposed engaging the support wall and one or the other of the forward internal wall and the rearward internal wall; the driven motive member being disposed adjacent and operative to engage one or the other of the rearward internal wall and the forward internal wall in opposite disposition relative to the bias member. Aspects of the above include the movable block having: a forward internal wall defined within the cavity, the forward wall being disposed near the raceway surface; and a rearward internal wall defined within the cavity defined further from the raceway surface than the forward internal wall; a support wall operatively associated with and disposed within but not structurally connected to the movable block, the support wall not being movable relative to the movability of the movable pressure block; the bias member being disposed engaging the support wall and the forward internal wall; the driven motive member being disposed adjacent and operative to engage the rearward internal wall in opposite disposition relative to the bias member; the bias member providing a Normally Closed disposition. Aspects of the above include the driven motive member being operatively disposed within the cavity to move the movable pressure block away from the biased Normally Closed position. Aspects of the above include the movable block having: a forward internal wall defined within the cavity, the forward wall being disposed near the raceway surface; and a rearward internal wall defined within the cavity defined further from the raceway surface than the forward internal wall; a support wall operatively associated with and disposed within but not structurally connected to the movable block, the support wall not being movable relative to the movability of the movable pressure block; the bias member being disposed engaging the support wall and the rearward internal wall; the driven motive member being disposed adjacent and operative to engage the forward internal wall in opposite disposition relative to the bias member; the bias member providing a Normally Open disposition. Aspects of the above include the driven motive member being operatively disposed within the cavity to move the movable pressure block away from the biased Normally Open position. Aspects of the above include the driven motive member being: operatively disposed within the cavity to move the movable pressure block away from the biased Normally Open or Normally Closed position. Aspects of the above include the driven motive member being: a pneumatic diaphragm operatively disposed within the cavity to inflate and move the movable pressure block away from the biased Normally Open or Normally Closed position. Aspects of the above include further comprising: the movable pressure block having at least one projection to engage and partially deform but not fully occlude a portion of tubing adjacent the rotor, the movable pressure block being movable to alternately engage and disengage the at least one projection and the race surface into or away from operative disposition relative to the rotor sub-assembly and the at least one roller at the partial arc of the rotor sub-assembly. Aspects of the above include further comprising: a moveable cover sub-assembly rotatably connected to the inlet and outlet guide members and operatively disposed to cover the operative engagement of the race surface and the arc of the rotor sub-assembly and a portion of each of the inlet channel and a portion of the outlet channel adjacent the race surface and the arc of the rotor sub-assembly; the moveable cover sub-assembly having at least one channel projection to enter a portion of either one or the other of the inlet and outlet channels to engage and partially deform but not fully occlude a portion of tubing adjacent the rotor. Aspects of the above include further comprising: an inlet guide member and an outlet guide member, the inlet guide member having an inlet tubing channel formed therein, the outlet guide member having an outlet tubing channel formed therein, the inlet guide member and the outlet guide member being disposed on opposite sides of the moveable pressure block and the inlet tubing channel and the outlet tubing channel forming substantially a linear form for receiving a tubing section in a substantially straight line.

Embodiments include a method for disposing a pump in one or the other of Normally Open or Normally Closed disposition, comprising: providing a movable pressure block to operatively engage a rotor sub-assembly having a partial arc in one or the other of Normally Open or Normally Closed disposition; the movable pressure block having an internal cavity defined therein and a race surface disposed thereon to contact with the rotor sub-assembly to fully occlude a portion of tubing adjacent the rotor, the movable pressure block being movable to alternately engage and disengage the at least one projection and the race surface into or away from operative disposition relative to the rotor sub-assembly and the at least one roller at the partial arc of the rotor sub-assembly; biasing within the cavity to hold the movable pressure block in one of the Normally Open position or Normally Closed position; and, driving within the cavity to overcome the holding of the bias member and move the movable pressure block away from the biased to Normally Open or Normally Closed position.

Embodiments include a blood component collection set, the blood component collection set comprising: a centrifuge to separate blood components from whole blood; an inlet tubing fluidly connected to a donor and the centrifuge; the centrifuge including a pump for one or more of biological, medical or intravenous fluids comprising: a base member; a rotor sub-assembly having at least one roller, the rotor sub-assembly being disposed connected to and supported by the base member, and the rotor sub-assembly defining a partial arc disposed to operatively engage a portion of the inlet tubing operatively engaged thereby; a movable pressure block having a race surface disposed thereon; and at least one projection to engage and partially deform but not fully occlude a portion of the inlet tubing adjacent the rotor, the movable pressure block being movable to alternately engage and disengage the at least one projection and the race surface into or away from operative disposition relative to the rotor sub-assembly and the at least one roller at the partial arc of the rotor sub-assembly; an inlet guide member and an outlet guide member, the inlet guide member having an inlet tubing channel formed therein, the outlet guide member having an outlet tubing channel formed therein, the inlet guide member and the outlet guide member being disposed on opposite sides of the moveable pressure block and the inlet tubing channel and the outlet tubing channel forming substantially a linear form for receiving a tubing section in a substantially straight line.

Embodiments include a blood component collection set, the blood component collection set comprising: a centrifuge to separate blood components from whole blood; an inlet tubing fluidly connected to a donor and the centrifuge; the centrifuge including a pump for one or more of biological, medical or intravenous fluids that is one of Normally Open or Normally Closed; the pump comprising: a base member; a rotor sub-assembly having at least one roller, the rotor sub-assembly being disposed connected to and supported by the base member, and the rotor sub-assembly having a partial arc disposed to operatively engage a portion of tubing operatively engaged thereby; a movable pressure block having a race surface disposed thereon to contact with the rotor sub-assembly to fully occlude a portion of tubing adjacent the rotor, the movable pressure block being movable to alternately engage and disengage the at least one projection and the race surface into or away from operative disposition relative to the rotor sub-assembly and the at least one roller at the partial arc of the rotor sub-assembly; the movable pressure block having a cavity defined therein, within which are disposed: at least one bias member disposed within the cavity to hold the movable pressure block in one of the Normally Open position or Normally Closed position; and, a driven motive member operatively disposed within the cavity to overcome the holding of the bias member and move the movable pressure block away from the biased Normally Open position or Normally Closed position.

Aspects of the above include an inlet tubing fluidly connected to a donor and pump; the inlet tubing being operatively loadable within the pump in operative association with the movable pressure block. Aspects of the above include a blood component collection set comprising: an inlet tubing fluidly connected to a donor and pump; the inlet tubing being operatively loadable within the pump in operative association with the movable pressure block.

Aspects of the above include a method for moving fluids through an apheresis system comprising: providing a pump, when drawing whole blood from a donor: receiving whole blood from a donor via an inlet tubing fluidly connected to and within the apheresis system; moving the whole blood through a draw pump to a centrifuge in the apheresis system; the draw pump being Normally Closed and having one or both a movable pressure block with at least one projection and a movable cover sub-assembly with at least one cover projection; moving components to collection with a return pump; the return pump being Normally Open and having one or both a movable pressure block with at least one projection and a movable cover sub-assembly with at least one cover projection.

Aspects of the above include further including an anticoagulant pump being Normally Closed. Aspects of the above include wherein the pump is part of a blood component collection set. Aspects of the above include wherein the blood component collection set is part of an apheresis system.

Embodiments include an apheresis system comprising: a needle inserted into a blood vessel of a donor to draw whole blood from the donor; a first tube having a lumen, fluidly associated with the needle, that moves the whole blood through the lumen; a draw pump engaged with the first tube that draws the whole blood into a centrifuge from the donor; the centrifuge that spins to cause centrifugal force to act on the whole blood to separate the whole blood into a least a first blood component and red blood cells; a blood component collection bladder, inserted into the centrifuge and fluidly associated with the first tube, that separates the first blood component from the whole blood; a second tube, fluidly associated the blood collection bladder, that moves the first blood component from the blood component collection bladder; a collection bottle, fluidly associated with the second tube, that extracts the first blood component from the apheresis system; a sensor positioned in physical proximity to the second tube to detect when a second blood component is being extracted from the whole blood; and after the second blood component is detected by the sensor and while the centrifuge continues to spin, a return pump, engaged with the second tube, that forces the separated first blood component back towards the blood component collection bladder through the second tube to move at least the red bloods cells from the blood component collection bladder and back into the donor; the draw pump being Normally Closed and having one or both a movable pressure block with at least one projection and a movable cover sub-assembly with at least one cover projection; moving components to collection with a return pump; and, the return pump being Normally Open and having one or both a movable pressure block with at least one projection and a movable cover sub-assembly with at least one cover projection. Aspects of the above include the apheresis system including wherein the first blood component is plasma and the second blood component is platelets, red blood cells, and/or high hematocrit blood. Aspects of the above include the apheresis system further comprising an anticoagulant pump to draw anticoagulant from an anticoagulant bag and mix the anticoagulant with whole blood at a manifold or junction fluidly associated with the first tube.

Embodiments include a blood component collection set associated with an apheresis system comprising: a needle inserted into a blood vessel of a donor to draw whole blood from the donor; a first tube having a lumen, fluidly associated with the needle, that moves the whole blood through the lumen, wherein a draw pump engaged with the first tube draws the whole blood from a donor; a blood component collection bladder, inserted into a centrifuge and fluidly associated with the first tube, that separates the first blood component from the whole blood; a second tube, fluidly associated with the blood collection bladder, that moves the first blood component from the blood component collection bladder; and a collection bottle fluidly associated with the second tube that extracts the first blood component from the apheresis system, wherein a sensor is positioned in physical proximity to the second tube to detect when a second blood component is being extracted from the whole blood; and wherein, after the second blood component is detected by the sensor and while the centrifuge continues to spin, a return pump engaged with the second tube forces the separated first blood component back towards the blood component collection bladder through the second tube to move at least the red bloods cells from the blood component collection bladder and back into the donor the draw pump being Normally Closed and having one or both a movable pressure block with at least one projection and a movable cover sub-assembly with at least one cover projection; moving components to collection with a return pump; and, the return pump being Normally Open and having one or both a movable pressure block with at least one projection and a movable cover sub-assembly with at least one cover projection. Aspects of the above include wherein the first blood component is plasma and the second blood component is platelets, red blood cells, and/or high hematocrit blood. Aspects of the above include the blood component collection set further comprising an anticoagulant pump to draw anticoagulant from an anticoagulant bag and mix the anticoagulant with whole blood at a manifold or junction fluidly associated with the first tube. Aspects of the above include including wherein the draw pump is disengaged when the return pump forces the separated first blood component back towards the blood component collection bladder through the second tube to move at least the red bloods cells from the blood component collection bladder and back into the donor. Aspects of the above include wherein the blood component collection set is part of an apheresis system.

Embodiments includes a pump for fluids comprising: a base member; an inlet guide member and an outlet guide member, the inlet guide member having an inlet tubing channel formed therein, the outlet guide member having an outlet tubing channel formed therein, the inlet guide member and the outlet guide member being disposed on the base member and the inlet tubing channel and the outlet tubing channel forming substantially a linear form for receiving a tubing section in a substantially straight line; a moveable cover sub-assembly rotatably connected to the inlet and outlet guide members and operatively disposed to cover a portion of each of the inlet channel and a portion of the outlet channel, the moveable cover sub-assembly having at least one channel projection to enter a portion of either one or the other of the inlet and outlet channels to engage and partially deform but not fully occlude a portion of tubing adjacent the rotor.

Aspects of the above include the at least one projection of the moveable pressure block provides a tortuous fluid path within a tubing portion engaged thereby.

Aspects of the above include wherein the at least one projection of the moveable cover member providing a tortuous fluid path within a tubing portion engaged thereby.

Aspects of the above include wherein the at least one projection of the moveable cover member includes at least first and second projections, the first projection being defined operatively insertable within the inlet tubing channel of the inlet guide member and the second projection being defined operatively insertable within the outlet tubing channel of the outlet guide member, both the first and second projections providing first and second tortuous fluid paths within a tubing portion engaged thereby. Aspects of the above include wherein the first projection and the second projection define a V-shaped surface configured to receive a tubing portion engaged thereby. Aspects of the above include wherein the V-shaped surface comprises chamfered edges. Aspects of the above include wherein one of the inlet channel and the outlet channel has at least a first recess defining a valley, and wherein the first recess, the first projection, and the second projection cause an internal diamond shape to form in a tubing portion engage with the first recess, the first projection, and the second projection. Aspects of the above include wherein one of the inlet channel and the outlet channel has at least a first recess, the first recess being defined to operatively receive the at least one projection of the moveable cover member insertable within one of the inlet channel of the inlet guide member and the outlet channel of the outlet guide member, the first recess providing a tortuous fluid path within a tubing portion engaged thereby. Aspects of the above include wherein the inlet channel or the outlet channel has one or more of at least a second recess and a third recess, the second and third recesses being defined to operatively receive the at least one projection of the moveable cover member insertable within one of the inlet channel of the inlet guide member and the outlet channel of the outlet guide member, the second and third recesses providing a tortuous fluid path within a tubing portion engaged thereby. Aspects of the above include wherein the at least one projection of at least one of the movable cover sub-assembly is configured to minimize at least one of longitudinal movement of a tubing portion in the inlet channel, the outlet channel or the raceway and lateral movement of a tubing portion in the inlet channel, the outlet channel or the raceway.

Any one or more of the aspects/embodiments as substantially disclosed herein.

Any one or more of the aspects/embodiments as substantially disclosed herein. optionally in combination with any one or more other aspects/embodiments as substantially disclosed herein.

One or more means adapted to perform any one or more of the above aspects/embodiments as substantially disclosed herein.

The present disclosure can provide a number of advantages depending on the particular aspect, embodiment, and/or configuration. By maintaining the speed of rotation of a centrifuge while moving the unneeded blood components back to the donor, an apheresis procedure can be reduced in time, possibly by 30% or more. This increase in efficiency allows for faster and more comfortable donations. With faster donation times, a donation center can obtain more donations in a typical day, which increases productivity and revenue. Further, donors are more likely to return to donate again if the donation is faster. Having faster donations may also allow donation centers to attract donors using other donation centers with slower donation speeds.

These and other advantages will be apparent from the disclosure.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “donor,” as used herein, can mean any person providing a fluid, e.g., whole blood, to the apheresis system. A donor can also be a patient that also provides a fluid to the apheresis system temporarily while the fluid is processed, treated, manipulated, etc. before being provided back to the patient.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

The term “module” as used herein refers to any known or later developed hardware, software, firmware, artificial intelligence, fuzzy logic, or combination of hardware and software that is capable of performing the functionality associated with that element.

The terms “determine”, “calculate” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below

### BRIEF DESCRIPTION OF THE DRAWINGS

FIG. 1 shows a perspective view of an operating environment of an apheresis system in accordance with embodiments of the present disclosure;

FIG. 2A is a perspective view of the apheresis system shown in FIG. 1;

FIG. 2B is a first detail perspective view of a pump of an apheresis system in accordance with embodiments of the present disclosure;

FIG. 2C is a second detail perspective view of a pump of an apheresis system in accordance with embodiments of the present disclosure;

FIGS. 2D-2T3 provide perspective, plan, elevation and schematic views of alternative implementations of the present disclosure;

FIG. 2U is a detail perspective view of a fluid valve control system in accordance with embodiments of the present disclosure;

FIG. 3 is a flow chart of a method of fluid control in a pump; and

FIG. 4 is a flow chart of a method of fluid control through an apheresis system.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

Embodiments of the present disclosure will be described in connection with apheresis methods and systems. Embodiments below may be described with respect to separating blood components from whole blood. However, this example procedure is provided simply for illustrative purposes. It is noted that the embodiments are not limited to the description below. The embodiments are intended for use in products, processes, devices, and systems for pumping fluids. For example, embodiments disclosed herein may be used for separating any composite liquid. Accordingly, the present disclosure is not limited to separation of blood components from whole blood.

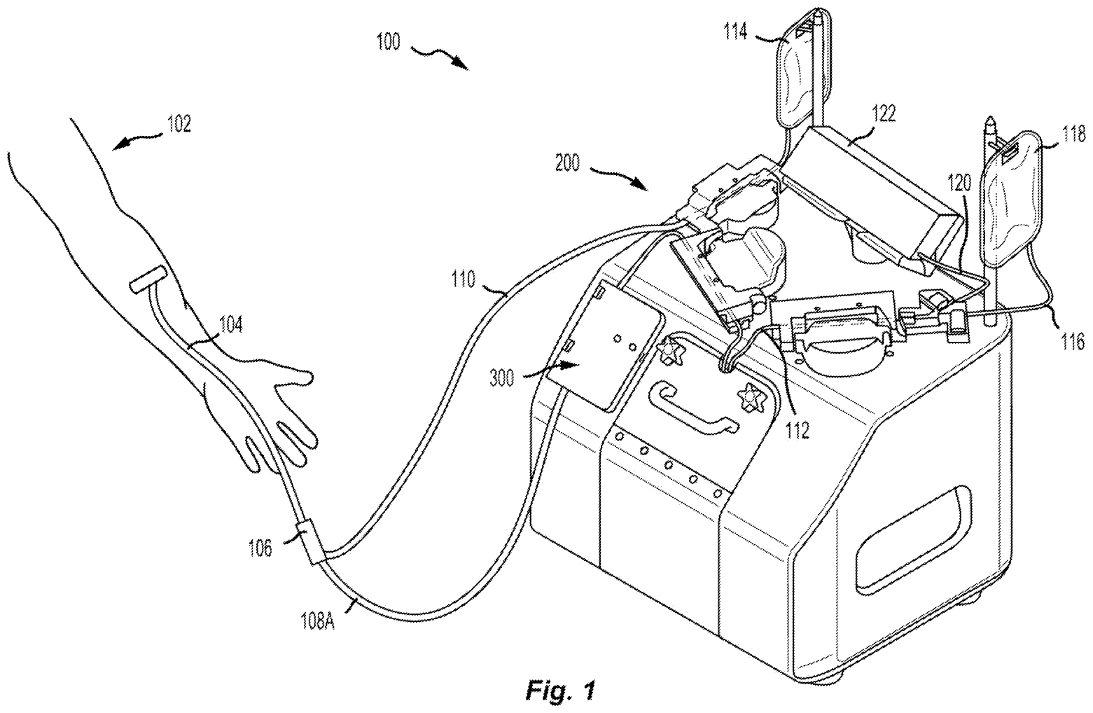

Referring to FIG. 1, a perspective view of an operating environment 100 of an apheresis system 200 is shown in accordance with at least one example embodiment of the present disclosure. The operating environment 100 may include an apheresis system 200, a donor 102, and one or more connections (e.g., donor feed tubing 104, cassette inlet tubing 108A, anticoagulant tubing 110, etc.) running from the donor 102 to the apheresis system 200, and/or vice versa. As shown in FIG. 1, the donor feed tubing 104 may be fluidly connected with at least one blood vessel, for example, a vein, of the donor 102 via venipuncture. For example, a cannula connected to an end of the donor feed tubing 104 may be inserted through the skin of the donor 102 and into a target site, or vein. This connection may provide an intravenous path for blood to flow from the donor 102 to the apheresis system 200, and/or for blood components to flow back to the donor 102. In at least one example embodiment, the fluid paths and connections may form an extracorporeal tubing circuit of the apheresis system 200.

Blood supplied from the donor 102 may flow along the donor feed tubing 104 through a tubing connector 106 and along the cassette inlet tubing 108A into a soft cassette assembly 300. The soft cassette assembly 300 may include one or more fluid control paths and valves for selectively controlling the flow of blood to and/or from the donor 102. The apheresis system 200 may include an anticoagulant supply contained in an anticoagulant (AC) bag 114. The anticoagulant may be pumped at least through the anticoagulant tubing 110 and the tubing connector 106 preventing the coagulation of blood in the apheresis system 200.

Anticoagulants can include one or more of, but are not limited to, citrate and/or unfractionated heparin. The AC bag 114 and other bags or bottles described herein can be made from, for example, one or more of, but not limited to: polyvinyl chloride (PVC), plasticized-PVC, polyethylene, ethylene with vinyl acetate (EVA), rubber, silicone, thermoplastics, thermoplastic elastomer, polymers, copolymers, and/or combinations thereof. The volume of AC in the AC bag 114 may vary based on the various factors, including the mass of the donor 102, the volumetric flow of blood from the donor, etc. In one example, the volume in the AC bag 114 may be 250 to 500 mL, although the volume in the AC bag 114 may be more or less than this volume.

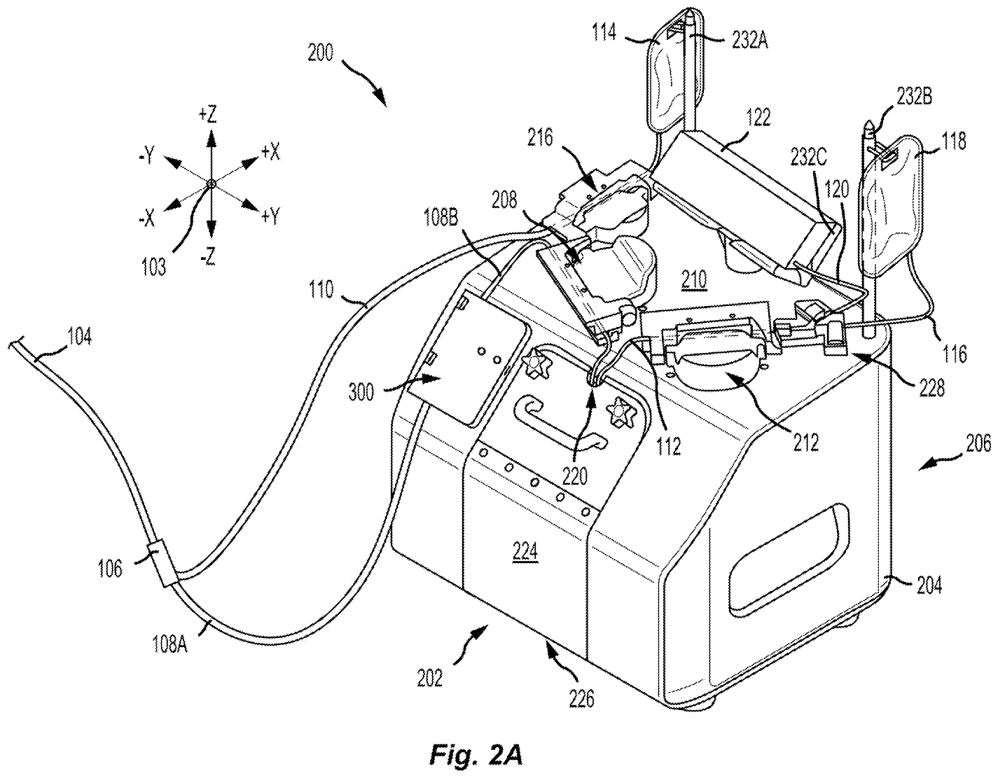

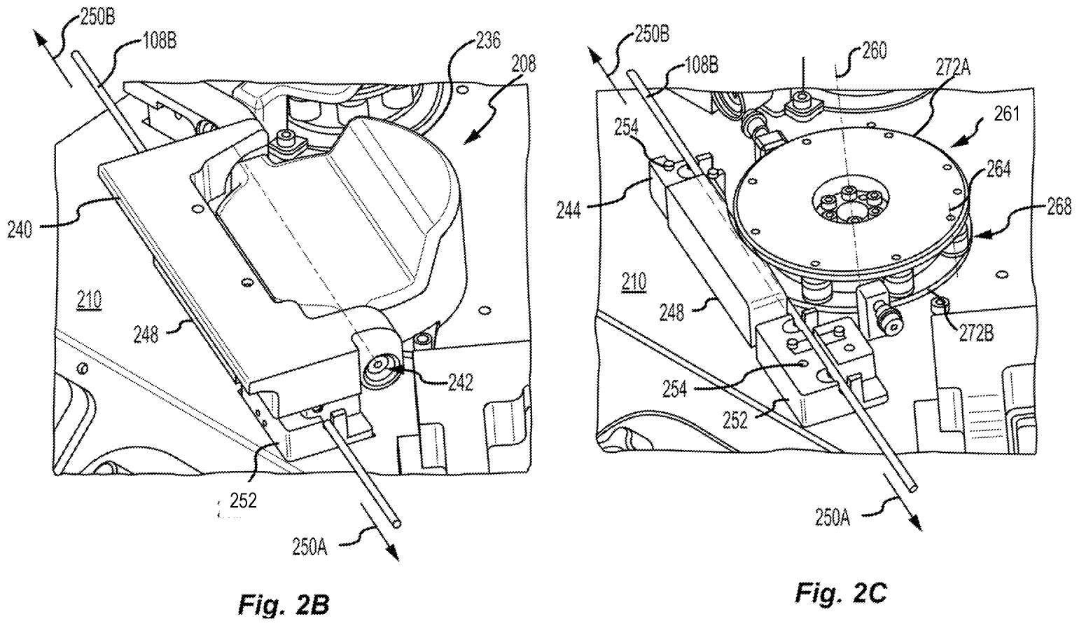

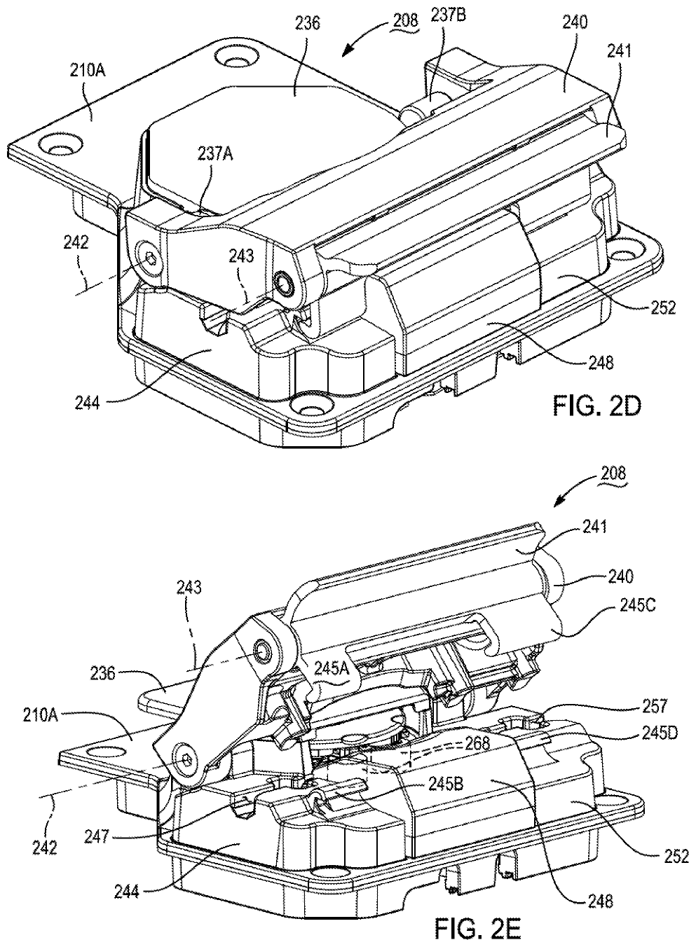

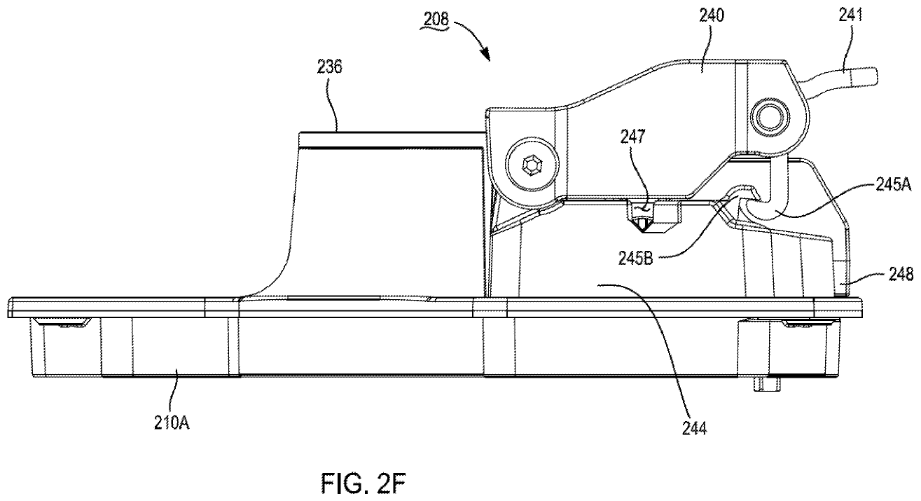

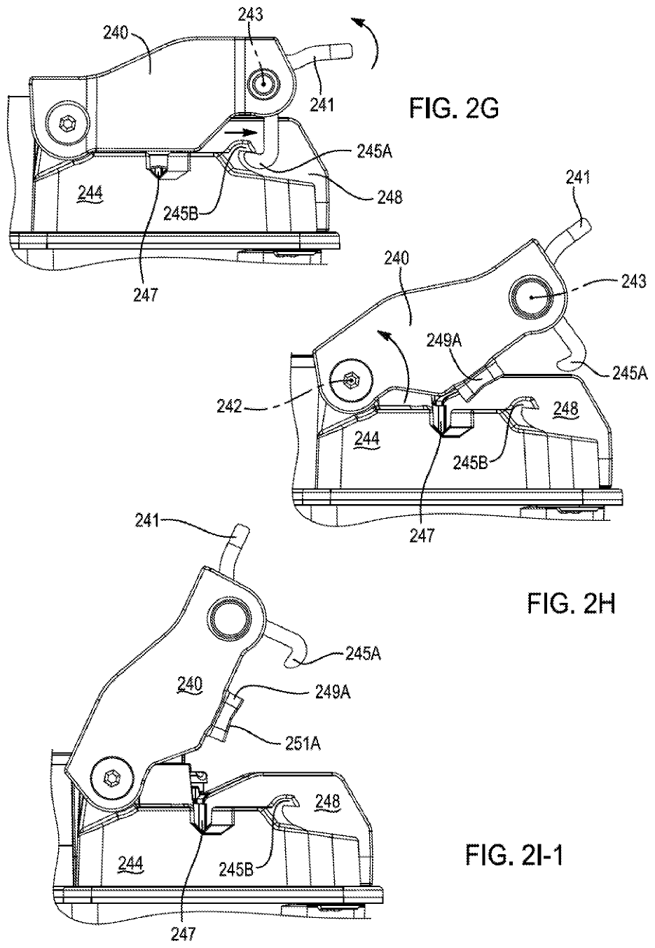

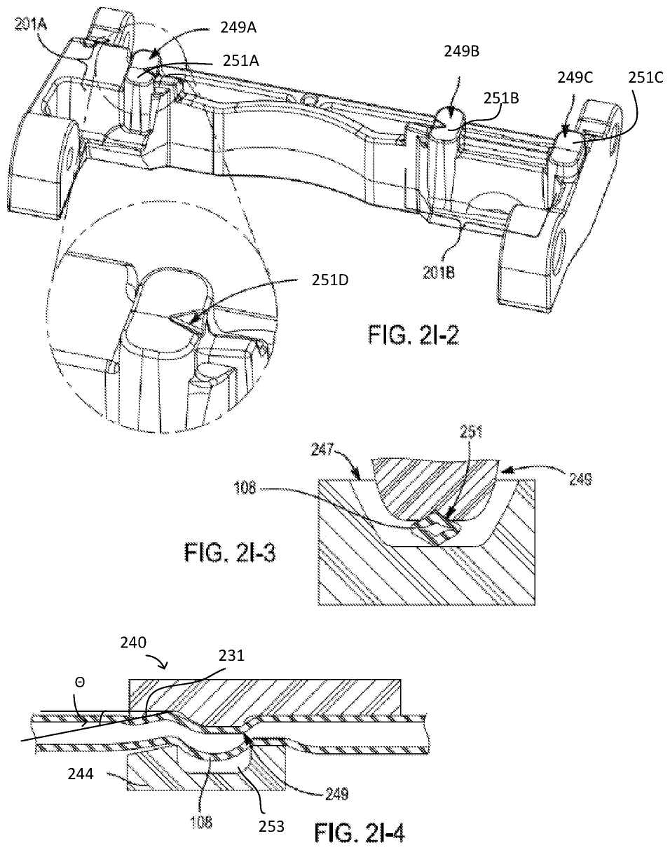

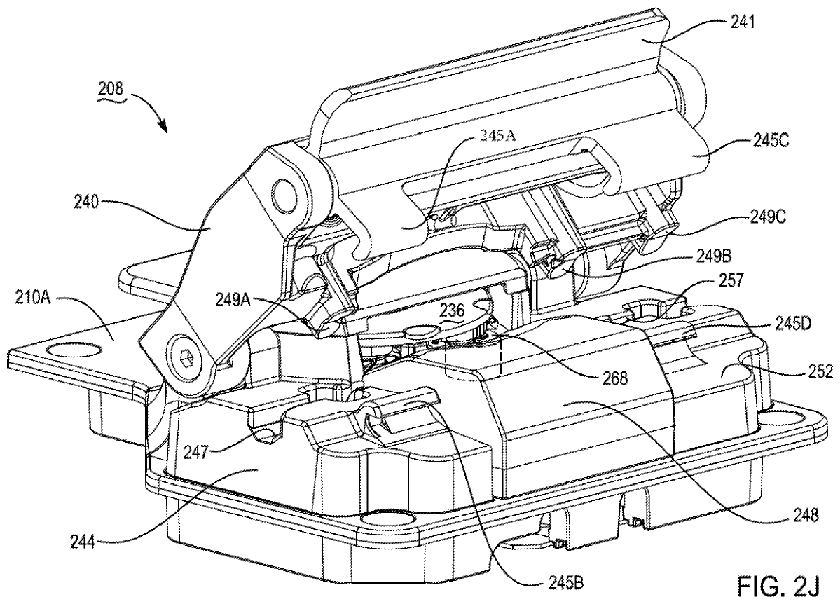

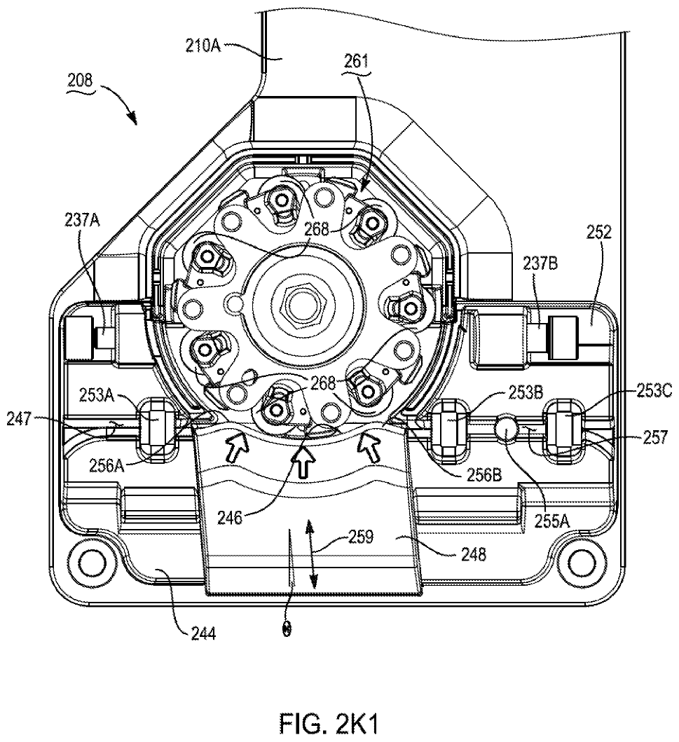

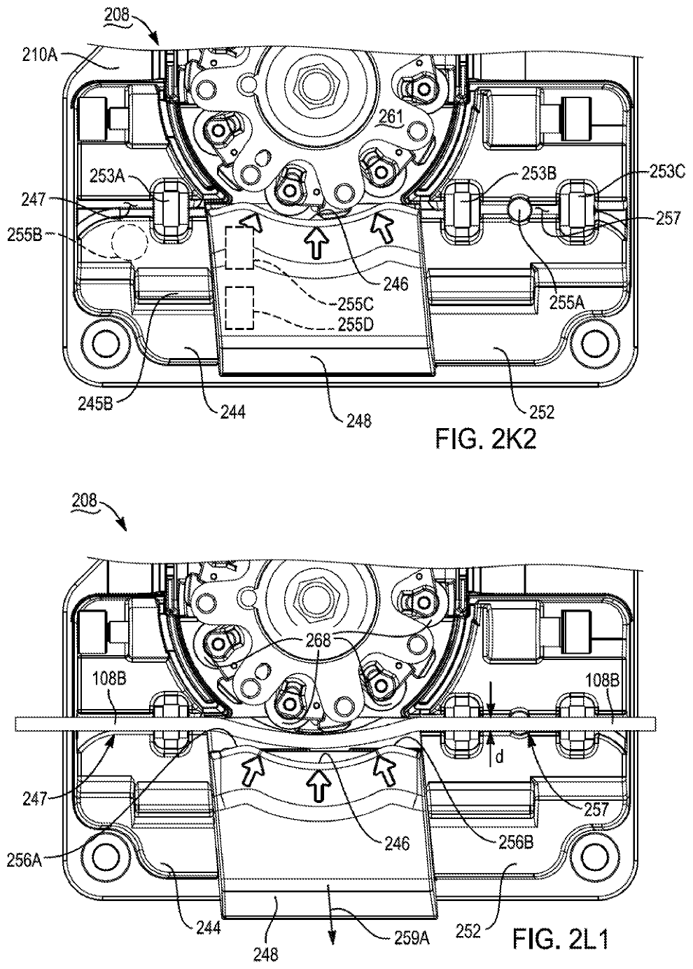

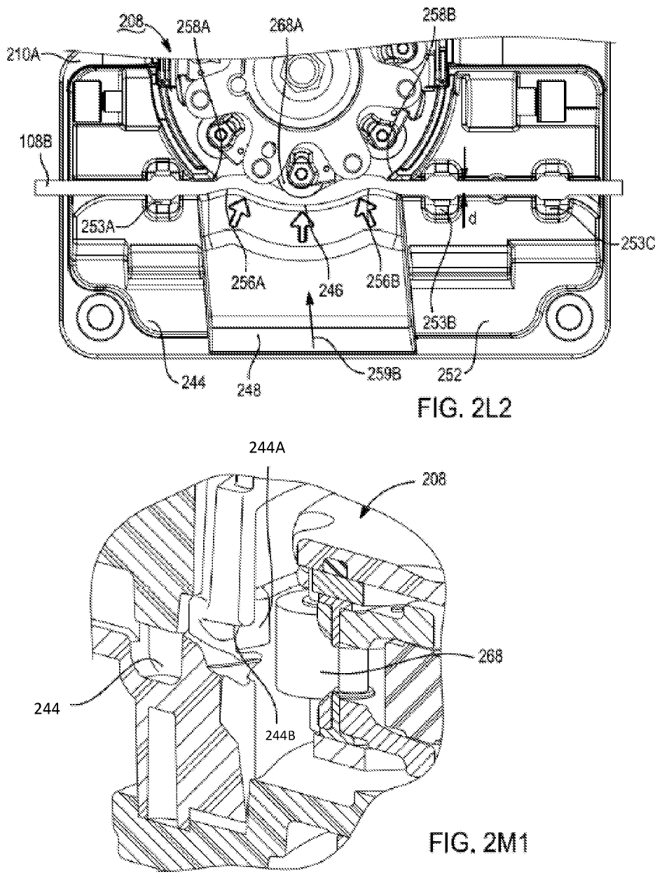

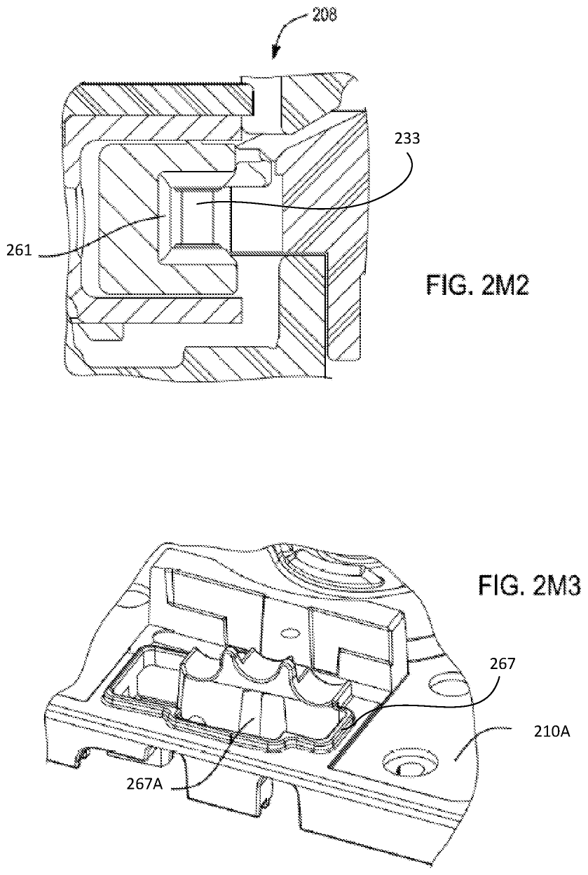

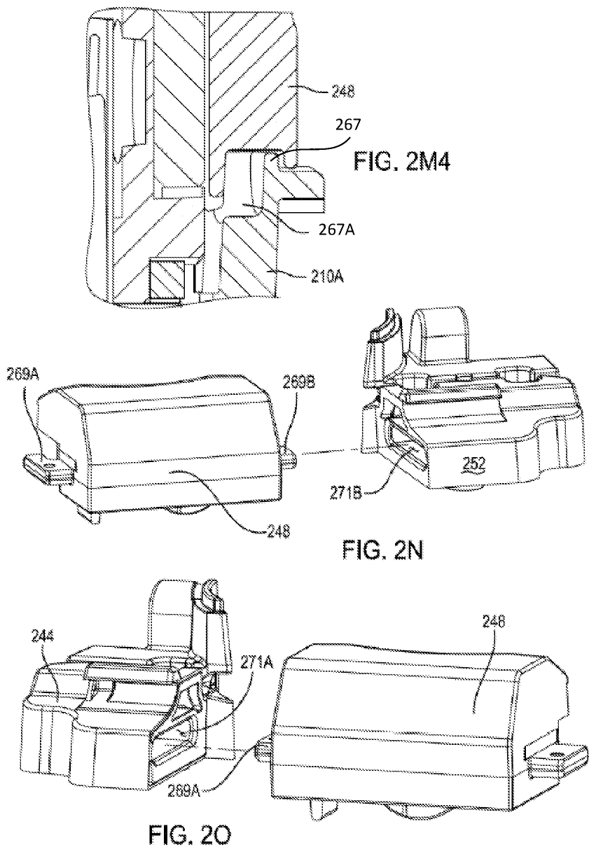

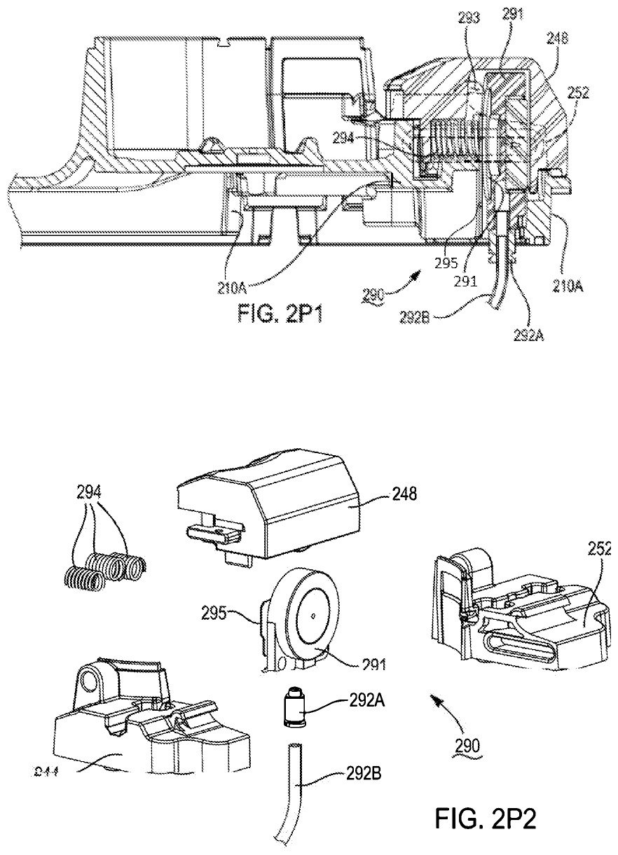

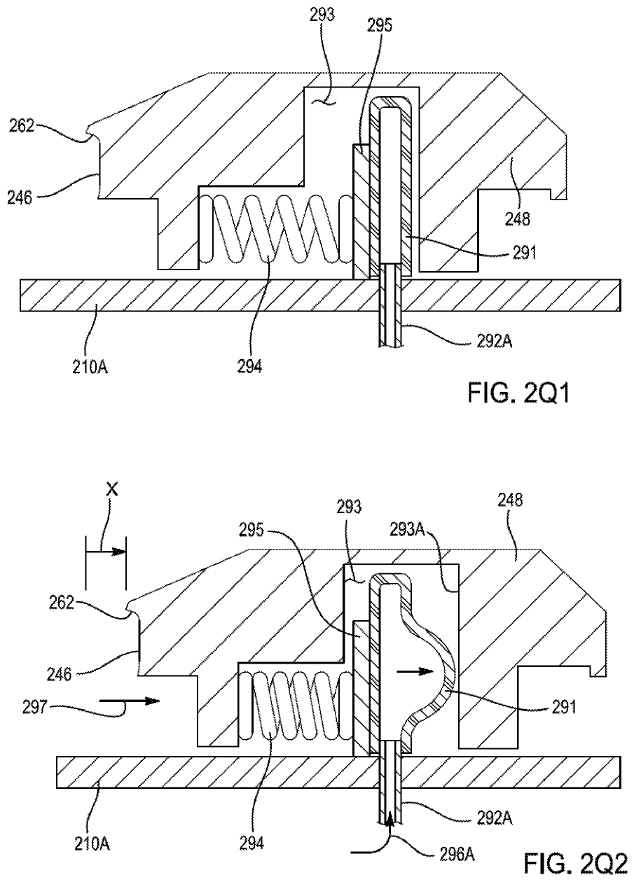

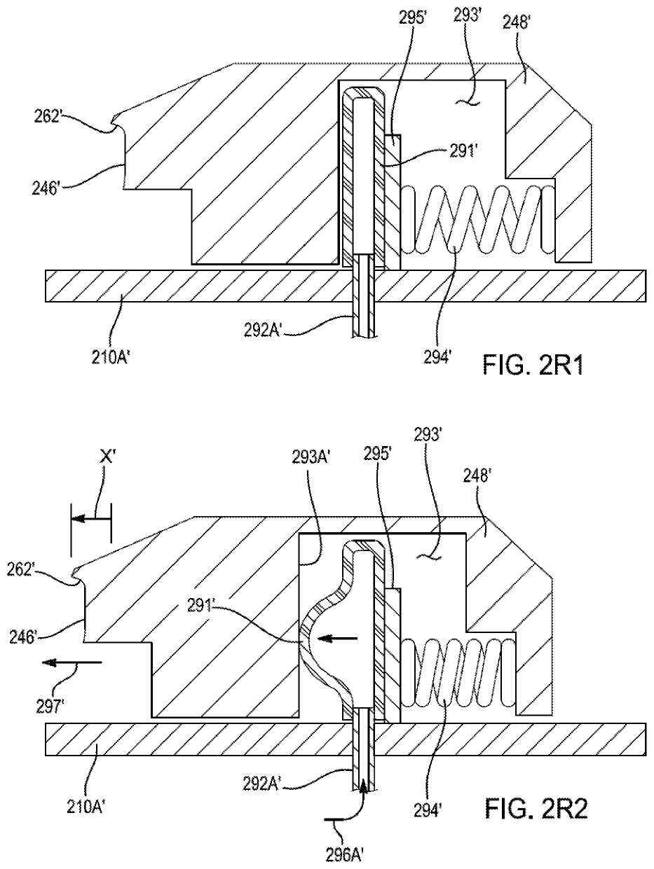

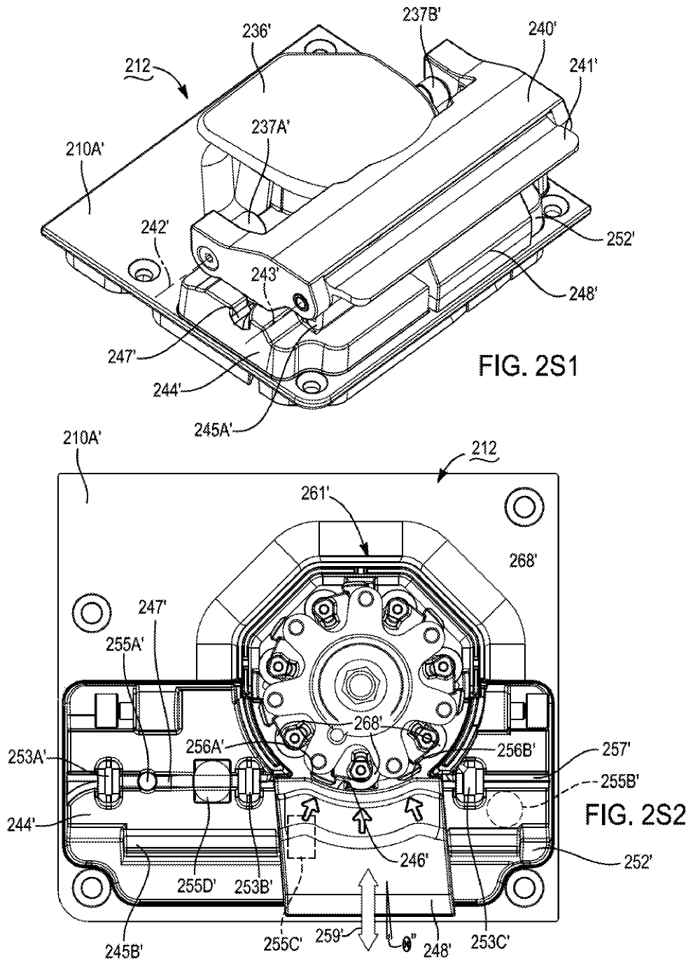

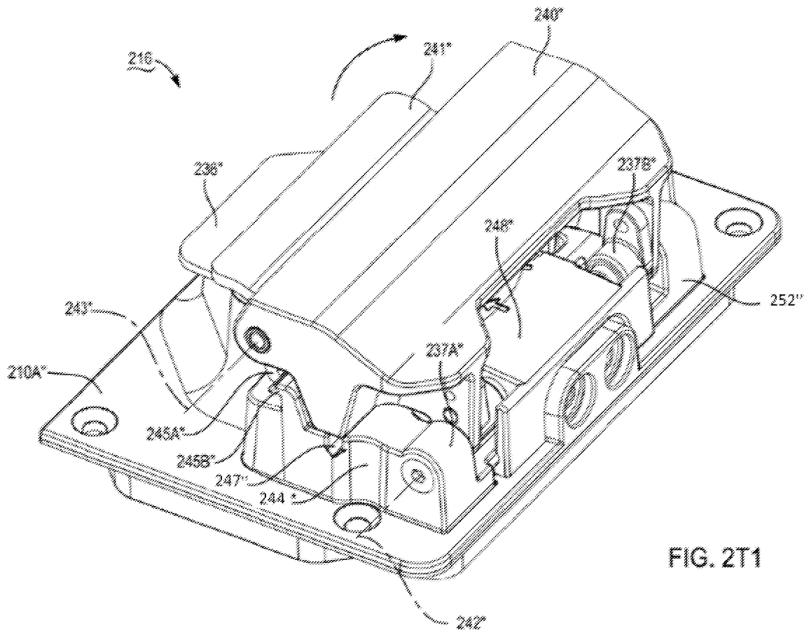

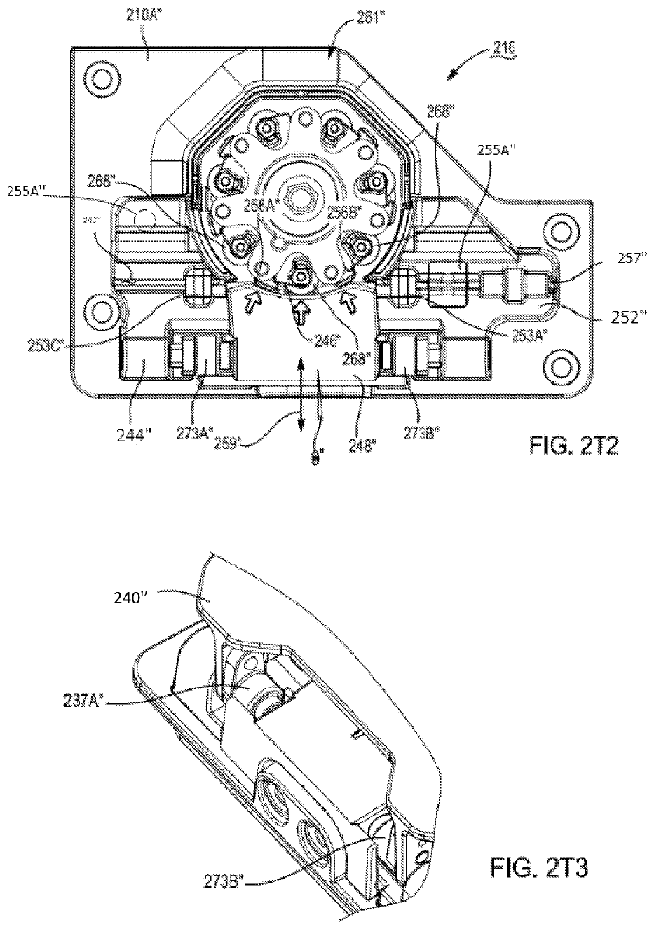

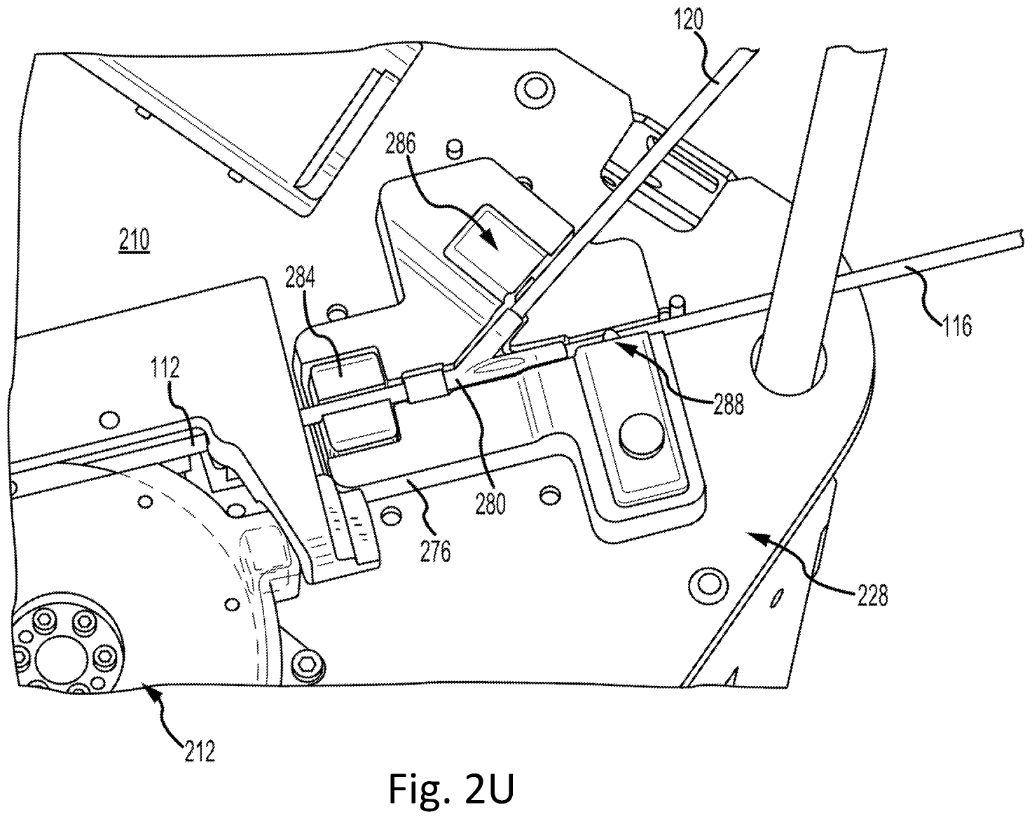

In at least one example embodiment, the apheresis system 200 may include a plasma collection bottle 122, or container, a saline fluid contained in a saline bag 118, and one or more lines or tubes such as saline tubing 116 and plasma tubing 120 (e.g., fluid conveying tubing, etc.) connecting the saline bag 118 and the plasma collection bottle 122 with the extracorporeal tubing circuit of the apheresis system 200. The amount of saline provided in the saline bag 118 can be 500 to 800 mL, although the volume in the saline bag 118 may be more or less than this volume. An example donation of a blood component, e.g., plasma, may be 880 mL. Thus, the plasma collection bottle 122 may hold at least this amount of plasma. In at least one example embodiment, the plasma collection bottle 122 may include a connection point disposed at, adjacent to, or in physical proximity to a substantially bottommost portion of the plasma collection bottle 122 (e.g., when the plasma collection bottle 122 is installed in a plasma collection cradle 232C, as shown in FIG. 2A). The connection point may include one or more connectors that are configured to interconnect with the plasma tubing 120 to receive and/or convey plasma. The disposition of the connection point at the bottom of the plasma collection bottle 122 can allow plasma contained in the plasma collection bottle 122 to move out of the plasma tubing 120 back through the lines, as described herein, without trapping air bubbles, etc. In at least one example embodiment, the plasma collection bottle 122 may be configured as a flexible bag, rigid container, and/or other container, and thus, the plasma collection bottle 122 is not limited to bottles or bottle-like containers.

FIG. 2A shows a perspective view of the apheresis system 200 described in FIG. 1. The apheresis system 200 may provide for a continuous whole blood separation process. In at least one example embodiment, whole blood may be withdrawn from a donor 102 and substantially continuously provided to a blood component separation device of the apheresis system 200 where the blood may be separated into various components and at least one of these blood components may be collected from the apheresis system 200. In at least one example embodiment, one or more of the separated blood components may be either collected, for subsequent use, or returned to the donor 102. The blood may be withdrawn from the donor 102 and directed into a centrifuge of the apheresis system 200 through an opening 220 in an access panel 224 of the apheresis system 200. In at least one example embodiment, the donor feed tubing 104, the cassette inlet tubing 108A, the inlet tubing 108B, the exit tubing 112, the saline tubing 116, and the plasma tubing 120, used in the extracorporeal tubing circuit may together define a closed, sterile, and disposable system, or blood component collection set, which may be further described hereinafter.

Examples of apheresis, plasmapheresis, and other separation systems that may be used with embodiments of the present disclosure, e.g., as apheresis system 200, include, but are not limited to, the SPECTRA OPTIA® apheresis system, COBE® spectra apheresis system, and the TRIMA ACCEL® automated blood collection system, all manufactured by Terumo BCT, of Lakewood, Colo.

Operation of the various pumps, valves, and blood component separation device, or centrifuge, may be controlled by one or more processors included in the apheresis system 200, and may advantageously comprise a plurality of embedded computer processors that are part of a computer system. The computer system may also include components that allow a user to interface with the computer system, including for example, memory and storage devices (RAM, ROM (e.g., CD-ROM, DVD), magnetic drives, optical drives, flash memory, etc.); communication/networking devices (e.g., wired such as modems/network cards, or wireless such as Wi-Fi); input devices such as keyboard(s), touch screen(s), camera(s), and/or microphone(s); and output device(s) such as display(s), and audio system(s), etc. To assist the operator of the apheresis system 200 with various aspects of its operation, in at least one example embodiment the blood component separation device, or centrifuge, may include a graphical user interface with a display that includes an interactive touch screen.

The apheresis system 200 may include a housing 204 and/or structural frame, a cover 210, an access panel 224 disposed at a front 202 and/or rear 206 of the apheresis system 200, and one or more supports 232A-C including hooks, rests, cradles, arms, protrusions, plates, and/or other support features for holding, cradling, and/or otherwise supporting a container or the AC bag 114, the saline bag 118, or the plasma collection bottle 122. In at least one example embodiment, the features of the apheresis system 200 may be described with reference to a coordinate system 103 and/or one or more axes thereof. The housing 204 may include a machine frame (e.g., made of welded, bolted, and/or connected structural elements, extruded material, beams, etc.) to which one or more panels, such as the cover 210, base members 210A (see FIGS. 2D-2T3, e.g.), doors, subassemblies, and/or components are attached. In at least one example embodiment, at least one panel of the apheresis system 200 may include a mounting surface for the soft cassette assembly 300, a first normally closed pneumatic pump such as a draw pump 208, a second normally closed pneumatic pump such as a return pump 212, a normally open pump such as an AC pump 216, and/or a fluid valve control system such as a fluid valve control system 228 (e.g., plasma and saline valve control, etc.).

The access panel 224 may include one or more handles, locks, and a pivoting or hinged axis 226 (e.g., a door hinge, piano hinge, continuous hinge, cleanroom hinge, etc.). In any event, the access panel 224 may be selectively opened to provide access to an interior of the apheresis system 200, and more specifically to a blood separation assembly, or centrifuge. In at least one example embodiment, the access panel 224 may provide access to load and/or unload the centrifuge with one or more components in the blood component collection set.

The inside of the apheresis system 200 may be separated into at least a centrifuge portion and a controls portion. For instance, the centrifuge portion may include a cavity configured to receive the centrifuge, rotation motor, and associated hardware. This area may be physically separated from the controls portion via one or more walls of the cavity. In at least one example embodiment, access to the controls portion (e.g., configured to house or otherwise contain the motor controller, CPU or processor(s), electronics, wiring, etc.) may be provided via a securely fastened panel of the housing 204, and/or panel separate from the access panel 224.

In at least one example embodiment, the apheresis system 200 may include, as shown in more detail in FIGS. 2A-2U, at least one of the draw pump 208, the return pump 212, or the AC pump 216 configured to control the flow of fluid (e.g., blood and/or blood components, anticoagulant, saline, etc.) to, through, and away from the apheresis system 200. For instance, as shown in FIG. 2A generally, the apheresis system 200 may include the draw pump 208 that controls blood flow to and/or from the donor 102 into and/or from the centrifuge of the apheresis system 200. The draw pump 208 may engage with a portion of the inlet tubing 108B disposed between the soft cassette assembly 300 and the centrifuge of the apheresis system 200. In at least one example embodiment, the apheresis system 200 may include the return pump 212 configured to control a flow of separated blood components (e.g., plasma, etc.) from the centrifuge via a tubing portion such as exit tubing 112 and/or the plasma tubing 120 to a plasma collection bottle 122 and/or vice versa. Additionally or alternatively, the return pump 212 may control a flow of saline (e.g., supplied from the saline bag 118 via tubing portions such as the saline tubing 116 and/or the exit tubing 112) throughout the blood component collection set and/or apheresis system 200. The AC pump 216 may engage with a portion of the anticoagulant tubing 110 to selectively control the flow of anticoagulant to the inlet tubing 104/108A throughout the blood component collection set of the apheresis system 200. As shown in FIG. 2A, the draw pump 208, the return pump 212, or the AC pump 216 can be disposed at least partially on a top portion of the cover 210 of the apheresis system 200, or may be connected to a base member 210A, a base member 210A′, or a base member 210A″, as shown in FIGS. 2D-2T3, inter alia, for otherwise attachment thereof to the cover 210 or the housing 204 or other in operative relation to and/or within apheresis system 200.

FIGS. 2B, 2C, 2D and 2E show various perspective views of the draw pump 208, the return pump 212, or the AC pump 216 the apheresis system 200 in accordance with at least one example embodiment of the present disclosure. The draw pump 208, the return pump 212, and the AC pump 216 may be described herein with reference to an apheresis system but it should be understood that they are not limited to use within an apheresis system. Although the draw pump 208 is shown and described, most specifically, in conjunction with FIGS. 2B-2M4, inter alia, it should be appreciated that the other pump assemblies of the apheresis system 200, i.e., the return pump 212 and the AC pump 216, may be different and operate differently in some particulars; however, in many instances the return pump 212 and/or the AC pump 216 may be or may include a substantially similar, if not identical, construction to the draw pump 208 described. For purposes of description, a draw pump 208 will be described first, with discussion of differences and other details for the return pump 212 and the AC pump 216 to come after. Elevation views of the exemplary draw pump 208 are shown in FIGS. 2F, 2G, 2H and 2I-1, with another perspective view in FIG. 2J followed by four plan views in FIGS. 2K1, 2K2, 2L1 and 2L2. Various details are apparent in some views and less so in others, with descriptions as follow.

As shown in several of FIGS. 2B-2J, the draw pump 208 may include a pump cover 236 or housing configured to at least partially enclose the moving elements of the draw pump 208. In at least one example embodiment, the draw pump 208 may include a hinged tubing guard door sub-assembly or a tubing guard 240 that is configured to open and close about a tubing guard pivot axis 242. In at least one example embodiment (not pictured herein), the tubing guard 240 may be attached to the pump cover 236 via one or more fasteners disposed along the tubing guard pivot axis 242, or as shown in another embodiment as in FIGS. 2D and 2K1, inter alia, guard 240 may instead be connected to respective guides such as an inlet guide 244 and an outlet guide 252 via rotational connections 237A and 237B. The door or the tubing guard 240 also holds down the tubing for sensors and to reduce or eliminate tubing movement. As shown in FIGS. 2B and 2C, blood provided by the donor 102 may be conveyed, or drawn, by the draw pump 208 into a centrifuge in a first draw or centrifuge direction 250A. Additionally or alternatively, blood or other fluid may be conveyed, or drawn, by the draw pump 208 toward the donor 102 in a donor direction 250B, opposite the centrifuge direction 250A.

In at least one example embodiment, the draw pump 208 and/or the return pump 212 and the AC pump 216 may be a tubing pump, peristaltic pump, diaphragm pump, and/or other pump configured to manipulate the flow of fluid (e.g., air, blood, blood components, anticoagulant, saline, etc.) in at least a portion of tubing. For example, the draw pump 208, the return pump 212, or the AC pump 216 may include a motor operatively interconnected with a rotating tubing contact assembly. In operation, the tubing (e.g., the inlet tubing 108B, the exit tubing 112, the anticoagulant tubing 110, etc.) may be inserted into the inlet guide 244, a tubing pressure block 248, and the outlet guide 252 adjacent to a rotating tubing contact head or a rotor sub-assembly 261 (e.g., FIG. 2C, 2K1 inter alia). In at least one example embodiment, the tubing pressure block 248 may be moved in a direction away from the rotating tubing contact head of the draw pump 208, the return pump 212, or the AC pump 216 providing a loading clearance area and/or allowing or providing for tubing to be either occluded or un-occluded when desired, or vice versa. The rotating tubing contact head or the rotor sub-assembly 261 (FIGS. 2C and 2K1, inter alia) may comprise a number of rotary pressure rollers 268 configured to rotate about respective pressure roller rotation axes 264 (FIG. 2C). Each of the rotary pressure rollers 268 may be disposed as shown in FIG. 2C between a first rotary plate 272A and a second rotary plate 272B of the rotor sub-assembly 261, where the first rotary plate 272A and the second rotary plate 272B may be configured to rotate about a pump rotation axis 260 and to maintain the rotary pressure rollers 268 in a desired Z-direction height. In at least one example embodiment, the first rotary plate 272A and the second rotary plate 272B may be a first end and a second end of the rotor sub-assembly 261 such that the first rotary plate 272A and the second rotary plate 272B are features of the rotor sub-assembly 261. In at least one example embodiment, the rotary pressure rollers 268 may be disposed at or near a periphery of the first rotary plate 272A and the second rotary plate 272B.

The one or more of the draw pump 208, the return pump 212, or the AC pump 216 may include, or operate similarly to, the Pulsafeeder® model UX-74130 peristaltic pump, Pulsafeeder® MEC-O-MATIC series of pumps, all manufactured by Pulsafeeder Inc., of Punta Gorda, Fla., without limitation. Other examples of the draw pump 208, the return pump 212, or the AC pump 216 may include, but are in no way limited to, the INTEGRA DOSE IT laboratory peristaltic pump manufactured by INTEGRA Biosciences AG, of Switzerland, and WELCO WP1200, WP1100, WP1000, WPX1, and/or WPM series of peristaltic pumps all manufactured by WELCO Co., Ltd., of Tokyo, Japan.

In general, once the tubing is loaded into the lead or the inlet guide 244 as shown for example in FIGS. 2C, 2L1, and/or 2L2, inter alia, the tubing pressure block 248, and/or the end or outlet or the outlet guide 252, at least some of the rotary pressure rollers 268 of the rotor sub-assembly 261 may be caused to engage with, contact, or otherwise compress the tubing disposed between the rotor sub-assembly 261 and the tubing pressure block 248. As the rotor sub-assembly 161 including the first rotary plate 272A and the second rotary plate 272B rotate about the pump rotation axis 260 the rotary pressure rollers 268 may compress a portion of the tubing between the rotor sub-assembly 261 of the draw pump 208, the return pump 212, or the AC pump 216 and the tubing pressure block 248 positively displacing fluid inside the portion of the tubing in a particular direction such as the centrifuge direction 250A or the donor direction 250B as the rotary pressure rollers 268 move. For instance, as the rotor sub-assembly 261 rotates in a counterclockwise direction about the pump rotation axis 260, the rotation of the rotary pressure rollers 268 compressing the tubing between the rotary pressure rollers 268 and the tubing pressure block 248 may displace, or pump, fluid in the centrifuge direction 250A. As another example, as the rotor sub-assembly 261 rotates in a clockwise direction about the pump rotation axis 260, the rotation of the rotary pressure rollers 268 compressing the tubing between the rotary pressure rollers 268 and the tubing pressure block 248 may displace, or pump, fluid in the donor direction 250B.

In at least one example embodiment, when the draw pump 208 is in a closed state and tubing is disposed between the rotor sub-assembly 261 and the tubing pressure block 248, at least one of the rotary pressure rollers 268 may be engaged with the tubing to fully occlude the tubing. As the rotor sub-assembly 261 rotates, the rotary pressure rollers 268 may move relative to the tubing pressure block 248 as described above. As shown in FIG. 2K1, the rotor sub-assembly 261 may be designed with seven rotary pressure rollers. This may ensure that when the draw pump 208 is closed, a portion of the tubing between the rotary sub-assembly 261 and the tubing pressure block 248 is fully occluded at all times which the rotary sub-assembly 261 is rotating. For example, as one of the rotary pressure rollers 268 is disengaging with a portion of tubing near the outlet guide 252, a rotary pressure roller near the inlet guide 244 may be engaging with the tubing to maintain a portion of the tubing that is fully occluded. In other embodiments, the rotor sub-assembly 162 may be designed with additional or fewer of the rotary pressure rollers 268 such that when the draw pump 208 is closed, a portion of the tubing between the rotary sub-assembly 261 and the tubing pressure block 248 is fully occluded.

When not actively pumping, the draw pump 208 can be maintained in a state where at least one of the rotary pressure rollers 268 continues to occlude the inlet tubing 108B (normally closed or NC) or in a state where none of the rotary pressure rollers 268 occludes the inlet tubing 108B (normally open or NO). Thus, the draw pump 208, based on the state when motionless, can also act as a “valve” to prevent (in NC) or allow (in NO) fluid movement. One or the other of these abilities may be available with one or more of the draw pump 208, the return pump 212, or the AC pump 216. For example, when the apheresis system 200 is operating, if the draw pump 208 is closed, the return pump 212 may be in an open state. Similarly, if the return pump 212 is closed, the draw pump may be in an open state.

The tubing guard 240 and the pump cover 236 may serve to protect an operator (e.g., phlebotomist, apheresis technician, etc.) and/or the donor 102 from incidental contact with one or more moving parts of the draw pump 208, the return pump 212, or the AC pump 216. In at least one example embodiment, the tubing guard 240 may be held in a closed position via one or more guard closure features 254 (see FIG. 2C) or a hook 245A, a hook 245B, a hook 245C or a hook 245D (see FIGS. 2D-2J, 2S1 and/or 2T1, inter alia) disposed in or in operative relation to the tubing guard 240, the inlet guide 244, the tubing pressure block 248, and/or the outlet guide 252. In some cases, these guard closure features 254 may be magnets (FIG. 2C) or springs contained in the tubing guard 240, the lead or the inlet guide 244, the tubing pressure block 248, and/or the end or the outlet guide 252. In at least one example embodiment, the draw pump 208, the return pump 212, or the AC pump 216 may be stopped or prevented from moving/operating when the tubing guard 240 is open. In this embodiment, a door closure sensor 255B, e.g., see FIG. 2K2, and/or 255B′ in FIG. 2S2, may be included in one or more of the guard closure features 254, the inlet guide 244 (in FIG. 2K2), and the outlet guide 252 (for 255B′ in FIG. 2S2), and/or the tubing pressure block 248.

In at least one example embodiment, some more and/or alternative pump details may be as follow. For example, in FIGS. 2D-2J, an exemplar draw pump 208 is shown with an exemplar door or the tubing guard 240 as this would cover a tubing section (not shown in FIGS. 2D-2J) in relation to the inlet guide 244 and the outlet guide 252, and the tubing pressure block 248 and the rotor sub-assembly 261 (under the pump cover 236). The door or the tubing guard 240 is shown herein particularly including a latch-hook sub-system. In particular a latch bar 241 is shown as disposed as part of and/or connected to a tubing cover or the tubing guard 240. In these examples, the latch bar 241 is pivotal in relation to the tubing guard 240 at and/or about the axis 243.

FIGS. 2E, 2F, 2G, 2H and 2I-1 show an exemplary opening process for the tubing guard 240 relative to the inlet guide 244 and the outlet guide 252 and the tubing pressure block 248. Starting, however, from a closed position as shown in FIGS. 2D and 2F, where it can be seen that the hook 245A on or connected to the latch bar 241 is initially engaged with the hook 245B of the inlet guide 244. Similarly, the hook 245C and the hook 245D are shown in FIG. 2E for and in relation to the outlet guide 252. In FIG. 2G, the latch bar 241 is shown as it is rotated, counterclockwise here but not limited thereto, to disengage the hook 245A and the hook 245B from each other. This rotation is about the axis 243. Then, the door or tubing guard 240 can be rotated, here also counterclockwise but not limited thereto, about the tubing guard pivot axis 242 as shown in FIG. 2H (see also 2E, e.g.) and then continued to a substantially open position as shown in FIGS. 2I-1 and 2J. This rotation of the tubing guard 240 is via the rotational connections 237A and 237B to the respective one of the inlet guide 244 and the outlet guide 252 as in FIG. 2D. An opposite direction rotation of both the tubing guard 240 and latch bar 241 would result in closure of the tubing guard 240 relative to the tubing, tubing guides, pressure block and rotor sub-assembly when the tubing is loaded. The latch bar 241 can be spring-loaded to be biased toward the un-rotated position of FIGS. 2F and 2H, 2I-1, and 2J for example after the external force applied in FIG. 2G is removed therefrom. In at least one example embodiment, the hook 245A may be manually engaged by an operator to disengage the hook 245A from the mating hook 245B. In order to re-engage the hook 245A and the mating hook 245B, an operator may press down on the tubing guard 240. When a force is applied to the tubing guard 240, the tubing guard 240 may rotate about the tubing guard pivot axis 242 and the hook 245A may catch on the mating hook 245B such that the tubing guard 240 is in a closed state as shown in FIGS. 2D and 2F.

In relation to the loading of tubing into the pump, starting in FIG. 2E and as shown in varying degrees thereafter in FIGS. 2F-2M2, channels such as inlet channel 247 and outlet channel 257 corresponding to the inlet guide 244 and the outlet guide 252 will receive the tubing, as shown more particularly in FIGS. 2L1 and 2L2, a tubing section such as the inlet tubing 108B will be received or disposed therein in a substantially linear or in-line disposition, sometimes referred to as a straight loading of tubing. In at least one example embodiment, the straight loading of tubing may enable the use of pump tubing or pump header tubing which does not differ from the tubing used throughout the entire tubing set. Additionally, several other structural and procedural features can make the present embodiments amenable to the use of pump tubing or pump header tubing which does not differ from the tubing used throughout the entire tubing set. More specifically, the present embodiments may ensure that different tubing is not necessary for the portion of tubing engaged within the rotor and race engagement area. Using a consistent type of tubing may result in lower costs of manufacture and parts procurement because in previous systems specially manufactured tubing was preferred or mandatory for use in/with the pump inasmuch as plasticizer amounts and strictly managed tubing thicknesses were needed for appropriate minimum operability and safety. In at least one example embodiment, the tubing used herein may be manufactured from material such as silicone or a different tubing polymer. The embodiments described herein may additionally increase the life of the tubing. As described herein, the present embodiments structurally and procedurally provide managed forces on the tubing that varies based on the tubing itself, the relative thicknesses and plasticized amounts thereof.

As introduced above and described further below, the inlet channel 247 and the outlet channel 257 introduced in FIGS. 2E and 2F are configured for and adapted to receive the inlet tubing 108B (FIGS. 2L1 and 2L2). Additionally, the tubing guard 240 has projections 249A, 249B and 249C (introduced in FIGS. 2H, 2I-1 and 2J) that contact with the inlet channel 247 and the outlet channel 257 to engage and at least partially contribute to holding the inlet tubing 108B in place.

Referring to FIGS. 2I-2-2I-4, each of the projections 249A, 249B, and 249C of the tubing guard 240 may include contoured surfaces 251A, 251B and 251C on the respective projections 249A, 249B, and 249C. The contoured surfaces 251A, 251B, and 251C may assist with engagement of the tubing 108B. In at least one example embodiment, the contoured surface 251A, 251B, and 251C may be relatively curved or may be upside-down V-shaped surfaces. In at least one example embodiment, at least one of the contoured surfaces 251A, 251B, and 251C may include a notch 251D. The projections 249A, 249B, and 249C, the contoured surfaces 251A, 251B and 251C, and the notch 251D (see FIGS. 211 to 214) may be disposed to enter and reside within respective recesses 253A, 253B and 253C, see e.g., FIGS. 2K1 and 2K2, of the inlet guide 244 and the outlet guide 252. In at least one example embodiment, the inlet block side 201A may include one projection 249 and the outlet block side 201B may include two projections 249. It will be appreciated that in other embodiments, the inlet block side 201A and the outlet block side 201B may include one projection, two projections, or more than two projections.

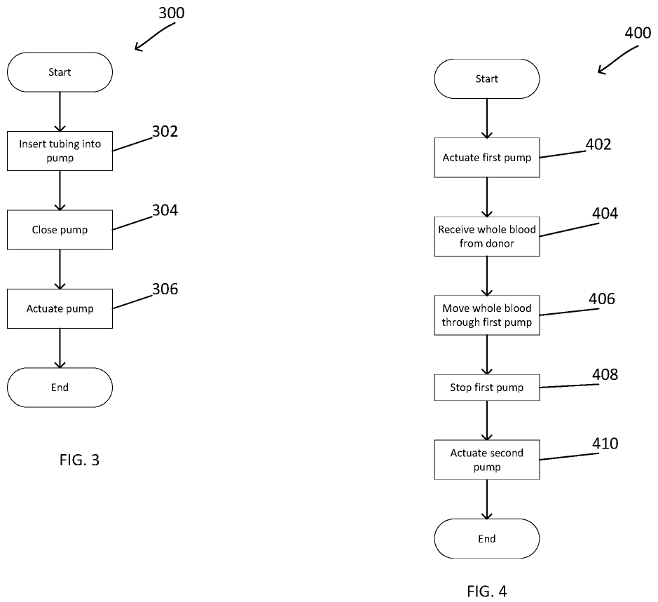

When the tubing guard 240 is closed relative to the inlet guide 244 and the outlet guide 252, the projections 249A, 249B, and 249C with the contoured surfaces 251A, 251B, and 251C may enter their respective recesses 253A, 253B, 253C, and engage and compress, without fully occluding, any tubing disposed therewithin. The engagement hereof may be partially occlusive such that fluid flow through the inlet tubing 108B is slightly impacted. This may provide a tortuous path but may not fully occlude the inlet tubing 108B. As shown in FIG. 2I-3, when the tubing guard 240 is closed relative to the inlet guide 244 and the outlet guide 252, the inlet tubing 108B may be clamped such that it is manipulated to have a substantially diamond shaped cross section. The engagement is mostly constraining and providing a holding function to reduce or eliminate movement of the tubing within the draw pump 208; particularly in the raceway/pump roller engagement area (described further below).

As shown in FIG. 2I-4, the tubing guard 240 may create an angle θ with the inlet guide 244 and the outlet guide 252. The angle θ in conjunction with a downward sloping portion 231 may help to point the inlet tubing 108B down as it enters the draw pump 208 and guide the inlet tubing 108B into a portion of the draw pump 208 such that the inlet tubing 108B is in a location of the draw pump 208 to be occluded by the rotary pressure rollers 268.

For purposes of description, the draw pump 208 of FIGS. 2K1, 2K2, 2L1 and 2L2 is shown without the pump cover 236 and the tubing guard 240. When the draw pump 208 is shown without the pump cover 236, the rotor sub-assembly 261 with several of the rotary pressure rollers 268 is exposed. Exposed also for greater clarity are a race or a raceway 246, the inlet channel 247, and the outlet channel 257 and associated recesses 253A, 253B and 253C as shown particularly in FIGS. 2K1 and 2K2. Greater details are also shown for the tubing pressure block 248 which in this and various implementations is movable in both directions 259 in and out relative to the rotor sub-assembly 261. This movement will be described further below.

In some embodiments, the inlet channel 247, the outlet channel 257, the inlet guide 244, the tubing pressure block 248 and the outlet guide 252 have in FIGS. 2K1 and 2K2 some sensors of note. In the outlet guide 252 and the outlet channel 257, a pressure sensor 255A is partially shown. In some example embodiments, the pressure sensor 255A may be a centrifuge pressure sensor (or CPS) that can engage the tubing in the outlet channel 257 to measure or determine the pressure of the fluid, here blood, therein; and, as this fluid is in pressure communication with the fluid within the centrifuge downstream hereof, the relative centrifuge pressure can be determined. Also shown in FIG. 2K2, schematically are a door closure sensor 255B and one or two moving block position sensors 255C and 255D. The door closure sensor 255B is disposed within inlet guide 244 and is operatively disposed, here near the hook 245B to sense the closure of the tubing guard 240. The door closure sensor 255B may be an inductive or other sensor. One or both of the moving block position sensors 255C and/or 255D may be, as here below the tubing pressure block 248 to sense closure (the moving block position sensor 255D) of the tubing pressure block 248 relative to the rotor sub-assembly 261 or alternatively an open disposition (the moving block position sensor 255C) of the tubing pressure block 248 relative to the rotor sub-assembly 261. One or both of the moving block position sensors 255C and/or 255D may be employed, and in some implementations these may be optical sensors triggered by the movement/disposition of the tubing pressure block 248.

The tubing pressure block 248 may additionally include a curved face of the raceway 246, disposed between a first projection 256A and a second projection 256B. The first projection 256A and the second projection 256B may be general transition zones of the raceway 246 that may be configured to guide the inlet tubing 108B from the inlet guide 244 to the curved face of the raceway 246 and into the outlet guide 252.

The raceway 246 may be disposed for operative substantial engagement with the rotary pressure rollers 268, which in sum is achieved by movement of the tubing pressure block 248 in directions 259 alternately bringing the raceway into and out of near engagement with the rotary pressure rollers 268 of the rotor sub-assembly 261. When in near engagement and with a rotor sub-assembly 261, a portion of the inlet tubing 108B that may be occluded by at least one of the rotary pressure rollers 268 may be disposed between the curved face of the raceway 246 and the rotor sub-assembly 261. In at least one example embodiment, the raceway 246 may provide a hard stop plate against which at least one of the rotary pressure rollers 268 can pinch or occlude the inlet tubing 108B against a surface of the raceway 246. In at least one example embodiment, a rolling motion of the roller can then move that occlusion either one way or the other along the tubing line to move fluid in the tubing relative to the moving occlusion. In at least one example embodiment, the rotor sub-assembly 261 may be configured to rotate in both a clockwise and a counterclockwise direction such that the occlusion can be moved in either direction along the inlet tubing 108B.

These functions and motions are shown for example in FIGS. 2L1 and 2L2. In FIG. 2L1, the tubing pressure block 248 is shown either moving or having moved in an outward direction 259A such that the raceway 246 slides open. In this position, there may be sufficient space between the rotary pressure rollers 268 and the surface of the raceway 246 to allow for placement of an un-occluded section of the inlet tubing 108B therebetween. The inlet tubing 108B is also shown being disposed in the inlet channel 247 and the outlet channel 257 of the inlet guide 244 and the outlet guide 252 respectively. The relatively un-occluded thickness or diameter dimension “d” is shown to illustrate schematically a substantially un-occluded disposition of the inlet tubing 108B throughout the residence of the tubing in and/or between the inlet guide 244, the outlet guide 252, the tubing pressure block 248 and the rotary pressure rollers 268.

As shown in FIG. 2L2 (in a slightly exaggerated fashion to illustrate the concepts) in moving the tubing pressure block 248 in an inward direction 259B, the raceway 246 engages the tubing and may squeeze it against a roller 268A (some thickness of the inlet tubing 108B remains to represent the thickness of the tubing sidewalls). In some example embodiments, the draw pump 208 may include sidewall features 258A and 258B that are typically not flow restraining when the inlet tubing 108B is engaged with the draw pump 208, but may help to maintain placement of the inlet tubing 108B within the draw pump 208. Additionally, the sidewall features 258A and 258B may serve to gradually lead the inlet tubing 108B into the raceway 246 so there is not a pressure spike when one of the rotary pressure rollers 268 contacts the raceway 246 to fully occlude the inlet tubing 108B. In at least one example embodiment, positioning of the inlet tubing 108B may create a tortuous path for the fluid but provides greater restraint in and/or for the tubing.

Additionally, as described above, the projections 249A, 249B, 249C (not shown in FIGS. 2L1 and 2L2) when brought down into and engaging within recesses 253A, 253B, 253C when the inlet tubing 108B is resident in the inlet channel 247 and the outlet channel 257, engage the inlet tubing 108B and squeeze it in a substantially non-occlusive manner in a relatively transverse direction. These are shown by slightly spread areas, diameters greater than “d”, of tubing within each of the recesses 253A, 253B, 253C of FIG. 2L2. These cooperate to hold the tubing substantially in place for the sensors and for the rotor pressure roller 268 engagement thereof. Also, noting again that the cooperation or coaction of one or more of these tubing holding features such as the inlet channel 247 and the outlet channel 257, with the recesses 253A, 253B, 253C and the projections 249A, 249B, 249C and/or the raceway 246 adjacent the rotary pressure rollers 268 and/or with the tubing pressure block 248, make for a straight loading of tubing and a possible use of a pump header section of the inlet tubing 108B to be the same tubing as any and or all of the other tubing in line throughout the tubing set. This may ensure that no specially developed tubing is necessary.

FIGS. 2M1-2M4 show additional views of the draw pump 208. FIG. 2M1 is a cross-sectional view of the apheresis system 200 showing additional details of the inlet guide 244. In at least one example embodiment, the inlet guide 244 may include a cut-out 244A that may be configured to provide a space for tubing to stretch which may prevent tubing from buckling within the apheresis system 200. In at least one example embodiment, the cut-out 244A may be a sidewall feature disposed at one or both ends of the raceway 246. For example, as tubing is fed through the apheresis system 200, it may stretch and contract as the pumps such as the draw pump 208 operate. As the inlet tubing 108B is stretched, it may increase in length which may tend to increase the likelihood of the tubing buckling. The cut-out 244A may be configured to enable the stretching of the tubing by providing an increased area for the tubing to reside which may prevent the tubing from buckling. The cut-out 244A may give the tubing additional room to expand into which may enable the use of a consistent tubing throughout the apheresis system. As described above, using a consistent tubing within the apheresis system 200 may decrease the costs associated with the apheresis system 200.

In some example embodiments, the inlet guide 244 may additionally include an overhang 244B that may be configured to assist in maintaining a desired position of tubing within the draw pump 208. For example, the overhang 244B may be configured to guide the inlet tubing 108B into the raceway 246 which may help to maintain the tubing within the raceway 246. It should be understood that features similar to the cut-out 244A and overhang 244B may be included in the outlet guide 252.

Referring to FIG. 2M2, a pivot shaft 233 of the rotor sub-assembly 261 is illustrated. In at least one example embodiment, a pivot shaft may be disposed between a pair of rotary pressure rollers 268.

Referring to FIG. 2M3 and FIG. 2M4, a fluid ingress prevention feature of the apheresis system 200 is shown. In at least one example embodiment, the fluid ingress prevention feature may be a levee 267 that mat surround an access point 267A to internal components of the draw pump 208. In at least one example embodiment, the levee 267 may be a feature of the base member 210A. The levee 267 may be prevent any fluid that may have escaped from any of the tubing through the apheresis system 200 from reaching electrical components of the pumps such as motors of the draw pump 208, the return pump 212, or the AC pump 216. In at least one example embodiment, the levee 267 may be disposed at a different location of the apheresis system 200 such that it is configured to capture any fluid prior to the fluid reaching any electrical components of the pumps of the apheresis system 200.

FIGS. 2N and 2O show two sub-part, partially exploded views of the tubing pressure block 248 in interaction with two other parts, the outlet guide 252 in FIG. 2N and inlet guide 244 in FIG. 2O. Here shown are two wing members 269A and 269B of the tubing pressure block 248 which are operably disposed in receiving slots 271A and 271B defined in the inlet guide 244 and the outlet guide 252 respectively. The receiving slots 271A and 271B are longer in the lateral direction, in and out of the page here, but corresponding to direction 259 of FIG. 2K1. This allows for the in and out movement of the tubing pressure block 248 relative to the rotor sub-assembly 261 as well as relative to the inlet guide 244 and the outlet guide 252. This also helps keep the tubing pressure block 248 from moving in any unwanted directions relative to the inlet guide 244 and the outlet guide 252 particularly when moving back and forth relative to the rotor and when the rotor is engaged in pressing against the raceway 246 in pumping motions.

In at least one example embodiment, a motive force that can be used to control movement of the tubing pressure block 248. Referring to FIG. 2P1, a pneumatic and spring system 290 is shown. FIG. 2P1 is a cross-sectional view showing the base member 210A and the tubing pressure block 248 with internal parts of the spring system 290. Here the spring system 290 is spring loaded and pneumatic and includes a diaphragm member 291, a pneumatic fitting 292A and pneumatic source tube 292B. The diaphragm member 291 is disposed inside a cavity 293 defined in the tubing pressure block 248. The pneumatic fitting 292A and the pneumatic source tube 292B may allow air and/or other gas or fluid to move therethrough to expand or contract the diaphragm. The spring system 290 may also include one or more compression springs 294 and a spring support structure 295. The spring support structure 295 may be proximate to or adjacent to the diaphragm member in at least one example embodiment. The one or more compression springs 294 and the spring support structure 295 are shown in more detail in FIG. 2P2 in an exploded view of the spring system 290 and the tubing pressure block 248. In FIG. 2P2, the inlet guide 244 and the outlet guide 252 are shown exploded to the sides and up and away from the tubing pressure block 248 with the spring system 290 exploded out below. The diaphragm member 291 is shown next to the pneumatic fitting 292A and the pneumatic source tube 292B with three compression springs 294 and the spring support structure 295. In other embodiments, there may be fewer than or more than three compression springs 294.

A schematic view of the diaphragm member 291, the pneumatic fitting 292A, and the one or more compression springs 294 is shown in FIGS. 2Q1 and 2Q2. In FIG. 2Q1, the spring system 290 is shown in a cross-sectional view with the base member 210A and the tubing pressure block 248. The tubing pressure block 248 shows here an upper lip 262 adjacent to the face of the raceway 246, the upper lip 262 may provide a shelf for holding the tube down against the face of the raceway 246 to maintain engagement with the rotary pressure rollers 268. The internal parts of the spring system 290 may include the diaphragm member 291 and the pneumatic fitting 292A. The diaphragm member 291 may be disposed inside the cavity 293 defined in the tubing pressure block 248. The one or more compression springs 294 and the spring support structure 295 may also be disposed within the cavity 293. Then, in FIG. 2Q2, air and/or gas and/or fluid is introduced at arrow 296A which expands the diaphragm member 291 to push on an interior wall 293A and move the tubing pressure block 248 to the open position away from the rotor sub-assembly 261 (not shown in FIG. 2Q2); a distance “x” is shown in FIG. 2Q2 to represent the differential movement of the tubing pressure block 248 in the direction 297 and compressing the one or more compression springs 294.

What is shown and described here is a Normally Closed (NC) operation wherein the tubing pressure block 248 is normally kept pushed forward by the one or more compression springs 294 toward the rotor sub-assembly 261 into the full engagement position. In at least one example embodiment, the full engagement position may be a position in which the face of the raceway 246 is disposed adjacent to the roller such that if a tubing were included, it would be fully compressed, in fully occluded disposition. The one or more compression springs 294 may maintain such position regardless of whether power is applied to the system. When the apheresis system 200 dictates that the tubing pressure block 248 should be opened, the pneumatic system may be initiated to power the diaphragm member 291 to move the tubing pressure block 248 to an open position against the NC, or Normally Closed condition. This can be and typically is a desired disposition for a draw pump, such as the draw pump 208 because this embodiment provides a safety catch or check valve operation so that in case of a power loss or accidental power shut down, no further blood is pulled or allowed to drain out of the donor and pushed or allowed back into the donor to whom this apheresis system 200 is connected to. If power is lost, the power is removed from solenoid valves that direct pressurized air to the diaphragm member 291 (e.g., pneumatic actuators). The actuators are vented to atmospheric pressure so that the actuators do not apply a force to the tubing pressure block 248 (e.g., races or raceways) which allows the one or more compression springs 294 move the tubing pressure block 248 to a desired, safe position (e.g., the NC position), as shown in FIG. 2Q1. With a normally closed pump at the NC position, the draw pump 208 acts like a closed valve and does not allow for any blood flow in the donor feed tubing 104, the cassette inlet tubing 108A, or the inlet tubing 108B.

Referring to FIGS. 2R1 and 2R2, a pump such as the return pump 212 is shown that is in a Normally Open, NO position in contrast to the draw pump 208 that is in a Normally Closed, NC, position. As shown in FIGS. 2R1 and 2R2, the return pump 212 may include springs such as one or more compression springs 294′ on the other side of a diaphragm, here diaphragm member 291′. In FIG. 2R1, the diaphragm member 291′ may be disposed inside a cavity 293′ defined in a tubing pressure block 248′. Also, a spring system 290′ is shown in a cross-sectional view with a base member 210A′ and the tubing pressure block 248′. The tubing pressure block 248′ may include an upper lip 262′ adjacent the face of a raceway 246′ the upper lip 262′ may be configured to hold a received tube down against the face of the raceway 246′. The internal parts of the spring system 290′ may include the diaphragm member 291′ and a pneumatic fitting 292A′. Also included are the one or more compression springs 294′ and a spring support structure 295′. As shown in FIG. 2R2, air and/or gas and/or fluid may be introduced at 296A′ which may expand the diaphragm member 291′ to push on an interior wall 293A′ and move the tubing pressure block 248′ to the closed position toward the rotor sub-assembly 261 (not shown in FIG. 2R2). The distance “x′” is shown as a distance that the tubing pressure block 248′ moves in the direction 297′ toward the rollers. The closed position here is under pneumatic power against the normally open, NO, disposition of the return pump 212. If pneumatic power is turned off and/or lost, the one or more compression springs 294′ may move the tubing pressure block 248′ back to a normally open position which allows for saline or plasma to move freely back to the centrifuge, depending upon the pressure difference if any.

Referring to FIGS. 2S1 and 2S2, the return pump 212 is shown. In at least one example embodiment, the return pump 212 may be similar to the draw pump 208. For example, in FIGS. 2S1 and 2S2, at least one example embodiment of the return pump 212 is shown with an exemplary door or tubing cover or tubing guard 240′ as this would cover a tubing section (not shown in FIGS. 2S1-2S2, but see the exit tubing 112 in FIG. 2A) in relation to the inlet guide 244′ and the outlet guide 252′, and the tubing pressure block 248′ and a rotor sub-assembly 261′ (under rotor cover 236′) on a base member 210A′. The door or tubing cover or the tubing guard 240′ is shown herein particularly including a latch-hook sub-system with a latch bar 241′ shown as disposed as part of and/or connected to a tubing cover or the tubing guard 240′. As before, the latch bar 241′ is pivotal in relation to the tubing guard 240′ at and/or about an axis 243′ while the tubing guard 240′ is pivotal about an axis 242′ relative to the inlet guide 244′ and the outlet guide 252′ and the rotor sub-assembly 261′. Rotational connections 237A′ and 237B′ provide this operative relationship. Similar to the structures and functions for the draw pump 208, a hook 245A′ on or connected to the latch bar 241′ is initially engaged with a mating hook 245B′ of the inlet guide 244′ and can be disengaged by a manual or similar external force applied to the latch bar 241′ as in FIG. 2G such that the tubing guard 240′ is opened as in FIGS. 2H and 2I. Reverse movement or action can be used to close the tubing guard 240′.

As shown in more detail in FIG. 2S2, the return pump 212 is shown without the rotor cover 236′ and the tubing guard 240′. Exposed then are the rotor sub-assembly 261′ with rotary pressure rollers 268′. Exposed also for greater clarity are a race or a raceway 246′, inlet channel 247′, outlet channel 257′, and associated recesses 253A′, 253B′ and 253C′ for holding the tubing in the pump and for the sensors. The return pump 212 also includes the tubing pressure block 248′, similar to the draw tubing pressure block 248 of the draw pump 208, which in this and various implementations may be movable in both directions of arrow 259′ relative to the rotor sub-assembly 261′. In at least one example embodiment, the tubing pressure block 248′ may not be wholly orthogonal to the tubing line, but, disposed at a slight angle Θ′ to provide an improved locational finder of the return pump 212. The raceway 246′ and the rotary pressure rollers 268′ may interact with each other to engage and hold a tubing section in operative relation within the return pump 212 substantially as described above with reference to the draw pump 208.

The inlet channel 247′, the outlet channel 257′, the inlet guide 244′, the tubing pressure block 248′, and the outlet guide 252′ of the return pump 212 may include one or more sensors. For example, as shown in FIG. 2S2, the inlet guide 244′ and the inlet channel 247′ may include a pressure sensor 255A′. The pressure sensor 255A′, also known as a centrifuge pressure sensor (or CPS) can engage the tubing in the inlet channel 247′ to measure or determine the pressure of the fluid, such as blood, therein; and, as this fluid is in pressure communication with the fluid within the centrifuge upstream hereof, the relative centrifuge pressure can be determined from the pressure sensor 255A′. Additionally, if the centrifuge pressure as determined from the pressure sensor 255A′ is sufficiently different from a pressure measured by the pressure sensor 255A of the draw pump 208, an alert may be triggered which may require an appropriate remedial action. Also shown in FIG. 2S2, schematically are a door closure sensor 255B′ and a moving block position sensor 255C′. In at least one example embodiment, the door closure sensor 255B′ may be similar to the door closure sensor 255B of the draw pump 208, but is here disposed within the outlet guide 252′ where it is operatively disposed to sense the closure of the door or the tubing guard 240′. The door closure sensor 255B′ may be an inductive or other sensor. In at least one example embodiment, the moving block position sensor 255C′ may be similar to the moving block position sensors 255C and/or 255D of the draw pump 208 and may be disposed under the tubing pressure block 248′ to sense closure of the tubing pressure block 248′ relative to the rotor sub-assembly 261′ (when it senses the presence of the tubing pressure block 248′) or alternatively an open disposition of the tubing pressure block 248′ relative to the rotor sub-assembly 261′ (when it senses an absence of the tubing pressure block 248′). In at least one example embodiment, the pressure sensor 255A′ may be similar to one or both of the moving block position sensors 255C and/or 255D of the draw pump 208 and in some implementations may be an optical sensor triggered by the movement/disposition of the tubing pressure block 248′.

In at least one example embodiment, the return pump 212 may additionally include an optical sensor 255D′ in or adjacent to the inlet channel 247′. The optical sensor 255D′ may be disposed on or in the inlet guide 244′ and/or the inlet channel 247′ to detect fluid, air, cellular concentration, color, and/or color change in the fluid coming from the exit tubing 112. For example, if red blood cells are sensed within the exit tubing 112, then saturation condition has been reached and the plasma collection may cease, at least temporarily and the pump or pumps may be reversed to move red blood cells back to the donor. The optical sensor 255D′ is preferably as close to the centrifuge as reasonable, and as shown in FIG. 2S2, may be disposed on the inlet side of the return pump 212.

The AC pump 216 is shown in more detail in FIGS. 2T1, 2T2, and 2T3, and though it has many similarities to the draw pump 208, it also has many distinctions because it is generally for pumping only anticoagulant AC and not a blood component or blood product. For example, the AC pump 216 may be configured to run only in one direction (though is not limited to do so) because it is configured to deliver or prevent delivery of AC to the inlet tubing line 104/108A (see FIGS. 1 and 2A). The AC pump 216 may also be configured to spin faster, and/or may be configured to be used with smaller tubing lines such as tubing with a smaller inside diameter (ID).

Additionally, the AC pump 216 may be a manual pump and may not have a pneumatic pressure block control that the draw pump 208 and the return pump 212 are equipped with. The AC pump 216 may generally still have passive spring, or like bias to keep the raceway in a normally closed disposition because it may not be optimal to have a free flow of AC into the donor 102 during a power off or outage condition.

Despite these distinctions, the AC pump 216 has some similarities to both the draw pump 208 and/or the return pump 212. For example, in FIGS. 2T1, 2T2, and 2T3, an AC pump 216 is shown with a door or tubing cover or tubing guard 240″ as this would cover a tubing section (not shown in FIGS. 2T1-2T3, but see the anticoagulant tubing 110 in FIG. 2A) in relation to the inlet guide 244′ and the outlet guide 252″, a tubing pressure block 248″, and a rotor sub-assembly 261″ (under rotor cover 236″) on a base member 210A″. The tubing guard 240″ is shown herein particularly including a latch-hook sub-system with a latch bar 241″ shown as disposed as part of and/or connected to the tubing guard 240″. As before, the latch bar 241″ is pivotal in relation to the tubing guard 240″ at and/or about an axis 243″ while the tubing guard 240″ is pivotal about an axis 242″ relative to the inlet guide 244″ and the outlet guide 252″ and the rotor sub-assembly 261″. Rotational connections 237A″ and 237B″ provide this operative relationship. Similar to the structures and functions for the draw pump 208 and the return pump 212, a hook 245A″ on or connected to the latch bar 241″ may be initially engaged with a mating hook 245B″ of a guide such as the outlet guide 252″ and can be disengaged by a manual or a similar external force applied to the latch bar 241″ as in FIG. 2G which may result in the tubing guard 240″ being opened similar to the operation shown in FIGS. 2H and 2I-1 with respect to the draw pump 208. In at least one example embodiment, a reverse movement or action can be used to close the tubing guard 240″.

FIG. 2T2 shows the AC pump 216 without a rotor cover 236″ and the tubing guard 240″. Without the rotor cover 236″ and the tubing guard 240″, the rotor sub-assembly 261″ and the rotary pressure rollers 268″ may be visible. Additionally, a race or a raceway 246″, inlet channel 247″ and outlet channel 257″, and associated recesses 253A″, 253B″ and 253C″ for holding the tubing in the pump and for the sensors are shown. Similar to the draw pump 208 and the return pump 212, the AC pump 216 include a tubing pressure block 248″ which in this and various implementations is movable in both directions of arrow 259″ relative to the rotor sub-assembly 261″. In at least one example embodiment, the tubing pressure block 248″ may not be orthogonal to the tubing line, but may be disposed at a slight angle Θ″ which may help to hold the tubing for the sensors and/or to counter the rolling motion and improve resistance to the raceway 246″ or the tubing pressure block 248″ or tubing movement. The raceway 246″ and the rotary pressure rollers 268″ may interact with each other and/or with projections 256A″ and/or 256B″ to engage and hold a tubing section in operative relation within the AC pump 216.

In at least one example embodiment, the AC pump 216 may include some difference from one or both of the draw pump 208 and the return pump 212. For example, the AC pump 216 may include the inlet channel 247″, the outlet channel 257″, the inlet guide 244″, the tubing pressure block 248″, the outlet guide 252″ and one or more additional structures and/or sensors of note. In at least one example embodiment, as shown in FIG. 2T2, the inlet guide 244″ and the outlet guide 252″ are flipped in orientation, such that the inlet guide 244″ is on the opposite side of the rotor sub-assembly 261″ and the rotary pressure rollers 268″ and the tubing pressure block 248″ as compared to the return pump 212. Similarly, the outlet guide 252″ may be disposed on an opposite side of the rotor sub-assembly 261″ and associated components. In at least one example embodiment, the inlet guide 244″ and the outlet guide 252″ may not be flipped in orientation and may be disposed in an orientation that is configured to interact with other relative operative devices of the apheresis system 200, principally of and as shown in FIGS. 1 and 2A.

Additionally, the AC pump 216 may include a first cam 273A″ and a second cam 273B″ that may be configured to assist in opening of a tubing guard 240 of the AC pump 216. As shown in FIG. 2T3, the first cam 273A″ and the second cam 273B″ may be coupled to or connected to the tubing guard 240″. Thus, the first cam 273A″ and the second cam 273B″ may enable opening of the tubing guard 240″ to access the AC pump 216. In at least one example embodiment, the first cam 273A″ and the second cam 273B″ may each include a surface that may couple with or intersect with the raceway 246″. The first cam 273A″ and the second cam 273B″ may move with the tubing guard 240″ to push the raceway 246″ into place. Thus, the raceway 246″ is only in place when the tubing guard 240″ is closed and is held in place by a spring force and the first cam 273A″ and the second cam 273B″.

In at least one example embodiment, the inlet guide 244″ and relative to inlet channel 247″ may include a first sensor 255A″ and/or a second sensor 255B″. The first sensor 255A″, may be an air or bubble detector that may be any light, ultrasonic, or other type of sensor that is configured to detect the presence of fluid and/or air in the anticoagulant tubing 110 of the AC pump 216. In at least one example embodiment, if the first sensor 255A″ detects air or another undesirable inclusion, an appropriate changed action may be triggered. In at least one example embodiment, if the first sensor 255A″ detects air or another undesirable inclusion, the AC pump 216 may be stopped. Also shown in FIG. 2T2, schematically is the second sensor 255B″ which may be a door closure sensor disposed within the inlet channel 247″. In at least one example embodiment, the first sensor 255A″ may be similar to the pressure sensors 255A and 255A′ of the draw pump 208 and the return pump 212. In at least one example embodiment, the second sensor 255B″ may be similar to the door closure sensor 255B of the draw pump 208 because the second sensor 255B″ may be disposed within the inlet guide 244″ where it is operatively disposed to sense the closure of the door or the tubing guard 240″. In at least one example embodiment, the second sensor 255B″ may be an inductive or other sensor as before. In at least one example embodiment, the AC pump 216 may include additional sensors (not shown). However, as the tubing pressure block 248″ is only passively forced by spring or like biassing, additional block sensors similar to the moving block position sensors 255C and/or 255D and/or 255C′ may not be included in the AC pump 216 but may be optionally included in at least one example embodiment.

In at least one example embodiment, because the tubing pressure block 248″ is passively forced into a normally closed position, the tubing pressure block 248″ may be opened by hand or another manual or external force pulling the tubing pressure block 248″ back from the rotor sub-assembly 261″ to load the anticoagulant tubing 110. Otherwise, it might be that a mechanical relationship may be achieved at or by the mechanical rotation open and upward of the tubing guard 240″ about the rotational connections 237A″ and 237B″ that this could lever the tubing pressure block 248″ backward and out away from the rotor sub-assembly 261″. This may be a good reason to have the tubing guard 240″ also oppositely oriented as shown by the arrow in FIG. 2T1, toward the tubing pressure block 248″ as opposed to away from it. The tubing guard 240″ may then also move the tubing pressure block 248″ away from the rotor sub-assembly 261 to open the raceway 246″ for loading the tubing when the tubing guard 240″ is opened. Then, during closing, force on the tubing guard 240″ may be relieved and a spring or similar passive bias may then push the raceway 246″ back into the operative normally closed disposition which may engage the tubing in relation to the rotor sub-assembly 261″ and the rotary pressure rollers 268″.

Referring to FIG. 2U, the fluid valve control system 228 of the apheresis system 200 is shown with one or more fluid control valves. In at least one example embodiment, one or more fluid control valves may be used to control the routing or flow direction of fluid conveyed throughout the tubing of the apheresis system 200. In at least one example embodiment, the apheresis system 200 may include the fluid valve control system 228 disposed adjacent to the saline bag 118 and/or the plasma collection bottle 122.

As shown in FIG. 2U, the exit tubing 112 may pass through the return pump 212 and interconnect with a saline and plasma tubing y-connector 280. The saline and plasma tubing y-connector 280 may allow connection of the exit tubing 112 to a saline tubing 116 line and a plasma tubing 120 line. The fluid valve control system 228 may include an air detection sensor 284 disposed at a first end of the saline and plasma valve housing 276 and surrounding a portion of the exit tubing 112. The air detection sensor 284 can be any light, ultrasonic, or other type of sensor that can detect the presence of fluid or air in the exit tubing 112 and provide that signal to a controller of the apheresis system 200. Types of air detection sensors that can be used as the air detection sensor 284 may include, for example, the SONOCHECK ABD05, made by SONOTEC US Inc., or another similar sensor.

The saline and plasma valve housing 276 may include a number of receiving features (e.g., grooves, channels, receptacles, etc.) that receive a portion of the exit tubing 112, the saline tubing 116, the plasma tubing 120, and/or the saline and plasma tubing y-connector 280. Upon detecting air in the exit tubing 112, the fluid valve control system 228 may selectively actuate one or more of the fluid control valves such as a plasma flow control valve 286 and a saline flow control valve 288. In at least one example embodiment, the detection of air via the air detection sensor 284 may be used to signal an operation step and/or trigger a step in a control method as described herein.

The plasma flow control valve 286 and/or the saline flow control valve 288 may be a solenoid valve, linear actuator, pinch valve, clamp valve, tubing valve, and/or other actuatable valve configured to selectively alter, e.g., occlude, a fluid passage associated with a particular portion of the exit tubing 112, the saline tubing 116, or the plasma tubing 120. As shown in FIG. 2U, the plasma flow control valve 286 may be configured to pinch a portion of the plasma tubing 120 at least partially contained in a receiving feature of the saline and plasma valve housing 276. The saline flow control valve 288 may be configured to pinch a portion of the saline tubing 116 at least partially contained in a receiving feature of the saline and plasma valve housing 276. In any event, the plasma flow control valve 286 and the saline flow control valve 288 may include an actuatable extendable finger that moves from a retracted, or partially retracted, position to an extended, or partially extended, position to pinch the portion of tubing contained in the saline and plasma valve housing 276. While the plasma flow control valve 286 and the saline flow control valve 288 may completely pinch the tubing (e.g., completely restricting fluid flow therethrough), it should be appreciated that the plasma flow control valve 286 and the saline flow control valve 288 may be partially actuated to a position that partially restricts fluid flow through a portion of the tubing.

Also described herein is a method 300 for fluid control in a pump. The method 300 may be described relative to the draw pump 208 but it should be understood that the method 300 may be performed with the return pump 212 and/or the AC pump 216. The method 300 may start and proceed to step 302 where tubing is inserted into a pump such as the draw pump 208. As described above, the pump may include an inlet guide such as the inlet guide 244 and an outlet guide such as the outlet guide 252. In at least one example embodiment, the tubing may be inserted into the draw pump 208 through the inlet guide 244, may rest between the raceway 246 and the rotor sub-assembly 261, and may exit through the outlet guide 252. The tubing pressure block 248 may be in a first position when the tubing is inserted into the draw pump 208.

Once the tubing is inserted into the pump, the method 300 may proceed to step 304 where the pump is closed. In some embodiments, closing the draw pump 208 may move the tubing pressure block 248 from the first position to a second position. Once the tubing pressure block 248 is closed, the tubing may be fully occluded between at least one of the rollers of the rotor sub-assembly 261 and the raceway 246 of the tubing pressure block 248.

Once the pump is closed, the method 300 may proceed to step 306 where the pump is actuated or operated. When the pump is actuated, the rotor sub-assembly 261 may begin to rotate such that at least one roller of the rotor sub-assembly 261 occludes the tubing to move fluid within the tubing in a direction corresponding to rotation of the rollers and the rotor sub-assembly 261.

In at least one example embodiment, the pump may further include the tubing guard 240 that may be configured to engage with the inlet guide and the outlet guide. For example, tubing disposed between the tubing guard 240 and the inlet guide 244 and tubing disposed between the tubing guard 240 and the outlet guide 252 may be clamped into a diamond shape. The tubing may enable fluid flow but may be secured in place between the inlet guide 244 and the tubing guard 240 and the outlet guide 252 and the tubing guard 240.

In at least one example embodiment, the tubing may be configured to expand or stretch when the pump is actuated. The inlet guide 244 and the outlet guide 252 may include at least one cut-out such as the cut-out 244A which may be configured to house the expanded tubing such that the tubing stays in place within the pump during operation of the apheresis system 200.

Also described herein is a method 400 of fluid control through an apheresis system such as the apheresis system 200. The method 400 may start and proceed to step 402 where a first pump is actuated to draw whole blood from a donor. In at least one example embodiment, the first pump may be the draw pump 208. The method 400 may then proceed to step 404 where whole blood from the donor is received via the inlet tubing 108B. The inlet tubing 108B may be fluidly connected to and within the apheresis system 200. The method 400 may then proceed to step 406 where whole blood is moved through the first pump of the apheresis system 200. In at least one example embodiment, the whole blood may be moved through the draw pump 208 and into a centrifuge of the apheresis system 200. As described above, in at least one example embodiment, whole blood may be moved through the draw pump 208 via the rotor sub-assembly 261. For example, the rotor sub-assembly 261 may begin to rotate such that at least one roller of the rotor sub-assembly 261 occludes the tubing to move fluid within the tubing in a direction corresponding to rotation of the rollers and the rotor sub-assembly 261.

In at least one example embodiment, the method 400 may continue to step 408 where the first pump is stopped. After the first pump is stopped, the method 400 may proceed to step 410 where the second pump may be actuated to move at least one component of the whole blood into a collection component of the apheresis system 200. In at least one example embodiment, the second pump may be the return pump 212. To actuate the second pump, the second pump may be moved from a normally open state to a closed state. When the second pump is moved from the normally open state to the closed state, the first pump may be moved from its normally closed state to an open state. Thus, when the apheresis system 200 is operational, only one of the first pump or the second pump may be closed at one time. This allows the first pump and the second pump to operate as valves for the apheresis system 200.

In at least one example embodiment, the apheresis system as described in the method 400 may additionally include a third pump. In at least one example embodiment, the third pump may be the AC pump 216 and may be in a normally closed state while the apheresis system 200 is operational.

The exemplary systems and methods of this disclosure have been described in relation to apheresis methods and systems. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the exemplary aspects, embodiments, and/or configurations illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices or collocated on a particular node of a distributed network, such as an analog and/or digital telecommunications network, a packet-switch network, or a circuit-switched network. It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

Also, while the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various aspects, embodiments, and/or configurations, includes components, methods, processes, systems and/or apparatus substantially as depicted and described herein, including various aspects, embodiments, configurations embodiments, sub combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A pump for fluids, the pump comprising:
a rotor sub-assembly, the rotor sub-assembly including at least one roller;
a tubing pressure block including a raceway and at least one projection, the tubing pressure block being movable between a first position and a second position;
an inlet guide including an inlet channel, the inlet guide disposed proximate to a first side of the tubing pressure block;
an outlet guide including an outlet channel, the outlet guide disposed proximate to a second side of the tubing pressure block, the second side of the tubing pressure block being disposed opposite the first side of the tubing pressure block such that there is a substantially straight path between the inlet guide and the outlet guide; and
a tubing guard configured to engage with the inlet guide and the outlet guide when the tubing guard is in a closed position and configured to expose at least a portion of the rotor sub-assembly, the tubing pressure block, the inlet guide, and the outlet guide when in an open position.

2. The pump of claim 1, wherein the raceway is curved and is configured to meet an arc of the rotor sub-assembly when the tubing pressure block is in the first position.

3. The pump of claim 1, wherein the pump is configured to engage a section of tubing, the section of tubing being disposed through the inlet guide, the raceway, and the outlet guide.

4. The pump of claim 3, wherein the section of tubing is configured to be occluded when the tubing pressure block is in the second position.

5. The pump of claim 4, wherein the at least one roller is configured to engage with the section of tubing to cause the tubing to be occluded when the tubing pressure block is in the second position.

6. The pump of claim 3, wherein the tubing guard comprises at least one downward sloped portion configured to guide tubing into the pump and at least one channel projection configured to engage with at least one of the inlet channel or the outlet channel when the tubing guard is in the closed position.

7. The pump of claim 6, wherein the at least one channel projection is configured to engage with and semi-occlude the section of tubing between the tubing guard and at least one of the inlet guide or the outlet guide when the tubing guard is in the closed position.

8. The pump of claim 6, wherein the at least one channel projection comprises a first channel projection configured to engage with the inlet channel and a second channel projection configured to engage with the outlet channel when the tubing guard is in the closed position.

9. The pump of claim 3, wherein the section of tubing is configured to stretch when the pump is in operation and wherein the raceway comprises at least one sidewall feature configured to collect a stretched portion of the section of tubing.

10. The pump of claim 1, further comprising at least one sensor disposed proximate to at least one of the inlet guide, the outlet guide, or the tubing pressure block.

11. The pump of claim 10, wherein the at least one sensor comprises at least one of a pressure sensor, a line sensor, a cover position sensor, a movable block position sensor, an inductive sensor, an optical sensor, a light sensor, an ultrasonic sensor, or an air or fluid sensor.

12. The pump of claim 1, wherein the tubing pressure block further comprises a cavity including at least one bias member configured to maintain the tubing pressure block in at least one of the first position or the second position.

13. The pump of claim 12, wherein the at least one bias member is at least one spring.

14. The pump of claim 12, wherein the tubing pressure block further comprises at least one driven motive member disposed within the cavity, the at least one driven motive member being configured to overcome the at least one bias member.

15. The pump of claim 14, wherein the at least one driven motive member is a pneumatic diaphragm, the pneumatic diaphragm configured to inflate and move the tubing pressure block away from the first position or the second position.

16. The pump of claim 15, wherein the pump is a normally closed pump, the first position is a closed position, the second position is an open position, the at least one bias member is configured to maintain the tubing pressure block in the first position, and the at least one driven motive member is configured to overcome the at least one bias member to move the tubing pressure block to the second position.

17. The pump of claim 15, wherein the pump is a normally open pump, the first position is a closed position, the second position is an open position, the at least one bias member is configured to maintain the tubing pressure block in the second position, and the at least one driven motive member is configured to overcome the at least one bias member to move the tubing pressure block to the first position.

18. The pump of claim 12, wherein the pump is an anticoagulant pump and the first position is a closed position.

19. The pump of claim 18, wherein the tubing pressure block is configured to move from the first position to the second position when an external force is applied to the tubing pressure block.

20. The pump of claim 1, further comprising a fluid ingress prevention feature configured to collect and prevent fluid from contacting at least one internal pump component.

21. A method for fluid control in a pump, the method comprising:
inserting tubing into a pump via an inlet guide and an outlet guide, the pump being in an open state with a tubing pressure block in a first position;
closing the pump by moving the tubing pressure block to a second position, the tubing being fully occluded between at least one roller of a rotor sub-assembly and a raceway of the tubing pressure block; and
actuating the pump including rotating the at least one roller such that fluid within the tubing is moved in a direction corresponding to rotation of the rollers.

22. The method of claim 21, wherein the pump further comprises a tubing guard configured to engage with the inlet guide and the outlet guide.

23. The method of claim 22, wherein the tubing is disposed between the tubing guard and the inlet guide and between the tubing guard and the outlet guide when the pump is closed.

24. The method of claim 23, wherein the tubing is clamped into a diamond shape when disposed between the tubing guard and the inlet guide and between the tubing guard and the outlet guide.

25. The method of claim 21, wherein the tubing is configured to expand when the pump is in operation and the inlet guide and the outlet guide include at least one cut-out configured to house the expanded tubing when the pump is in operation.

26. A method of fluid control through an apheresis system, the method comprising:
actuating a first pump to draw whole blood from a donor,
receiving whole blood from the donor via an inlet tubing fluidly connected to and within the apheresis system; and
moving the whole blood through the first pump of the apheresis system.
stopping the first pump; and
actuating a second pump to move at least one component of the whole blood to a collection component of the apheresis system including moving the second pump from an open state to a closed state,
wherein when the second pump is actuated, the first pump is moved from a closed state to an open state.

27. The method of claim 26, wherein the apheresis system further comprises:
a third pump that is configured to be in a closed state while the apheresis system is operational.
